(19) United States
(12) Patent Application Publication
(10) Pub. No.: US 2025/0311707 A1
(43) Pub. Date: Oct. 9, 2025

(54) TRANSGENIC MOUSE MODELS OF HUMAN ADAPTIVE AND INNATE IMMUNITY AND METHODS OF USE

(71) Applicant: The Jackson Laboratory, Bar Harbor, ME (US)

(72) Inventor: James Keck, Bar Harbor, ME (US)

(73) Assignee: The Jackson Laboratory, Bar Harbor, ME (US)

(21) Appl. No.: 18/866,819

(22) PCT Filed: May 18, 2023
(86) PCT No.: PCT/US2023/022662

§ 371 Date: Nov. 18, 2024

Related U.S. Application Data
(60) Provisional application No. 63/343,735, filed on May 19, 2022.
(60) Provisional application No. 63/343,747, filed on May 19, 2022.
(60) Provisional application No. 63/343,784, filed on May 19, 2022.
(60) Provisional application No. 63/343,799, filed on May 19, 2022.

(51) Int. Cl. A01K 67/0278

(52) U.S. Cl. CPC A01K 67/0278; A01K 2217/052; A01K 2227/105; A01K 2267/0325; A01K 2267/0393

(57) ABSTRACT

Provided herein are humanized immunodeficient mouse comprising human myeloid cells and human lymphoid cells, wherein the human lymphoid cells comprise human B cells that produce circulating immunoglobulin (Ig), for example, physiological levels of human IgG, and wherein the mouse expresses a detectable level of human FLT3L protein and does not express a detectable level of mouse FLT3 protein.

### RELATED APPLICATIONS

This application claims the benefit under 35 U.S.C. § 119(e) of U.S. provisional application No. 63/343,735, filed May 19, 2022, U.S. provisional application No. 63/343,747, filed May 19, 2022, U.S. provisional application No. 63/343,784, filed May 19, 2022, and U.S. provisional application No. 63/343,799, filed May 19, 2022, each of which is incorporated by reference herein in its entirety.

### BACKGROUND

Humanized mice contain human cell populations. Humanized mice are a robust in vivo platform for analyzing the safety and effectiveness of potential new drugs to modulate the immune system. They are also advantageous in vivo models for long-term studies in the fields of human immune cell biology, immuno-oncology, and infectious disease. Models engrafted with human cord blood-derived hematopoietic stem cells (HSC), for example, develop multi-lineage engraftment and display robust T-cell maturation and T-cell dependent inflammatory responses. In addition, an improved human myeloid and NK lineage development has been demonstrated in certain immunodeficient mouse models. As another example, models engrafted with human peripheral blood mononuclear cells (huPBMC) enable short-term studies requiring mature human T cells. HuPBMC mice are used as in vivo models to study and evaluate compounds for T cell immune modulation, infectious diseases and acute graft-versus-host disease (GVHD), which is a major problem in clinical hematopoietic stem cell transplantation. While these currently-available mouse models serve as invaluable tools for studying human innate immunity, none have been able to model a critical component of human adaptive immunity—none have been able to support human B cell development and maturation.

### SUMMARY

Provided herein are mouse models that support adaptive immune cell and human innate immune cell and development and maturation. The data provided herein demonstrates, unexpectedly, that certain immunodeficient mouse strains (e.g., NSG-Flt3^{null} (hu-FLT3L)), when engrafted with human peripheral mononuclear blood cells (huPBMCs), support expansion, development, and maturation of functional human B cells as well as human monocytes, T cells, NK cells, and all three subsets of dendritic cells (i.e., cDC₁, cDC₂, and plasmacytoid DC cells). Current huPBMC engrafted mouse models are unable to support expansion of myeloid cells and expansion mature B cells that are capable of class switching to produce physiological levels of human IgG. Surprisingly, the data provided herein obtained, for example, from huPBMCs engrafted NSG-Flt3^{null} (hu-FLT3L) mice, demonstrate successful production of a mouse model that can support a full range of major human immune cell populations, including dendritic cells, T cells, and functional mature B cells where IgG production is at human physiological levels (see data in Examples relating to IgG assay). A mouse that can support expansion, development, and maturation of this more complete repertoire of human immune cells will better model human immune responses and a myriad of clinical diseases in which functional B cells and dendritic cells are vital.

Some aspects of the present disclosure provide a humanized immunodeficient mouse comprising human myeloid cells and human lymphoid cells, wherein the human lymphoid cells comprise human B cells that produce circulating immunoglobulin (Ig), and wherein the genome of the mouse comprises (a) a null mutation in an endogenous Flt3 allele and (b) a nucleic acid encoding the human FLT3L protein.

Some aspects of the present disclosure provide a humanized immunodeficient mouse comprising human myeloid cells and human lymphoid cells, wherein the human lymphoid cells comprise human B cells that produce circulating immunoglobulin (Ig), optionally physiological levels of human IgG, and wherein the mouse expresses a detectable level of human FLT3L protein and does not express a detectable level of mouse FLT3 protein.

Other aspects of the present disclosure provide a humanized immunodeficient mouse comprising human myeloid cells and human lymphoid cells, wherein the human lymphoid cells comprise human B cells that produce circulating immunoglobulin (Ig), wherein the mouse is characterized by a severe combined immune deficiency mutation (scid), IL2 receptor gamma chain deficiency, MHC class I molecule deficiency (H2-K and D), MHC class II molecule deficiency (IA), FLT3 deficiency, and expression of human FLT3L protein. In some embodiments, the mouse comprises a NOD scid gamma mouse.

In some embodiments, the mouse is engrafted with about 1 million to about 5 million human peripheral blood mononuclear cells (huPBMCs), optionally about 1 million, about 2 million, about 3 million, about 4 million, or about 5 million huPBMCs.

In some embodiments, the mouse is engrafted with fewer than 5 million huPBMCs.

In some embodiments, the human B cells produce circulating human IgG.

In some embodiments, the human B cells produce at least 500 μg/ml IgG, at least 1000 μg/ml IgG, or at least 5500 μg/ml IgG.

In some embodiments, the human B cells produce circulating human IgM.

In some embodiments, the human B cells produce at least 0.5 μg/ml IgM, at least 1.5 μg/ml IgM, at least 2.5 μg/ml IgM, at least 5 μg/ml IgM, or at least 10 μg/ml IgM.

In some embodiments, the mouse further comprises human T cells, human NK cells, and human dendritic cells.

In some embodiments, the dendritic cells comprise the following subtypes: cDC₁, cDC₂, and plasmacytoid DC cells.

In some embodiments, bone marrow of the mouse comprises functional human B cells and functional human plasma cells.

In some embodiments, the mouse has undergone a myeloablative treatment and is deficient in mouse immune cells, optionally wherein the myeloablative treatment comprises a myeloablative chemical treatment or sublethal irradiation.

In some embodiments, the genome of the mouse comprises a null mutation in an endogenous Flt3 allele.

In some embodiments, the mouse is homozygous for a mouse Flt3^{null} allele.

In some embodiments, the genome of the mouse comprises a nucleic acid encoding the human FLT3L protein.

In some embodiments, the mouse has a non-obese diabetic (NOD) genetic background.

In some embodiments, the genome of the mouse comprises a null mutation in an endogenous Protein Kinase, DNA-Activated, Catalytic Subunit (Prkdc) allele, optionally a scid mutation in the endogenous Prkdc allele.

In some embodiments, the genome of the mouse comprises a null mutation in an endogenous Interleukin-2 Receptor Gamma (IL-2Rγ) allele.

In some embodiments, the genome of the mouse comprises a null mutation in an endogenous Recombination Activating Gene 1 (Rag1) allele.

In some embodiments, the mouse has a NOD scid gamma genetic background.

Some aspects provide a method of producing the humanized immunodeficient mouse of any one of the preceding paragraphs, the method comprising administering human peripheral blood mononuclear cells (huPBMCs) to an immunodeficient mouse, wherein the immunodeficient mouse expresses a detectable level of human FLT3L protein and does not express a detectable level of mouse FLT3 protein.

Other aspects provide method of producing the humanized immunodeficient mouse of any one of the preceding paragraphs, the method comprising: subjecting an immunodeficient mouse to a myeloablative treatment; and administering human peripheral blood mononuclear cells (huPBMCs) to the immunodeficient mouse, wherein the immunodeficient mouse expresses a detectable level of human FLT3L protein and does not express a detectable level of mouse FLT3 protein.

Yet other aspects provide a method of producing a mouse model of a human immune system, the method comprising: administering human peripheral blood mononuclear cells (huPBMCs) to an immunodeficient mouse, wherein the mouse expresses a detectable level of human FLT3L protein and does not express a detectable level of mouse FLT3 protein.

In some embodiments, the administering comprises administering at least 1 million of the huPBMCs to the immunodeficient mouse.

In some embodiments, the administering comprises administering 1 million to 20 million, 1 million to 15 million, 1 million to 10 million, or 1 million to 5 million of the huPBMCs to the immunodeficient mouse.

Some aspects provide a method comprising: administering a target drug to the humanized immunodeficient mouse of any one of the preceding paragraphs; and assaying a biological sample from the mouse for a characteristic of an anti-drug antibody (ADA) response.

In some embodiments, the method further comprises obtaining the huPBMCs from a human subject.

In some embodiments, the method further comprises predicting how the human subject will respond to the target drug based on the assaying of the biological sample from the mouse.

In some embodiments, the characteristic is selected from ADA titer, neutralizing capacity, binding affinity, and isotype.

In some embodiments, the target drug is selected from vaccines, antibodies, recombinant protein therapeutics (e.g., growth factors), cell-based therapies (e.g., chimeric antigen receptor (CAR)-T cell, TCR-engineered T cell, tumor-infiltrating lymphocyte (TIL), and regulatory T cell (Treg) therapies), DNA-based (e.g., gene, antisense oligonucleotide) therapies, and RNA-based (e.g., RNAi and mRNA) therapies.

Other aspects provide a method comprising administering a human therapeutic agent to the humanized immunodeficient mouse of any one of the preceding paragraphs; and assaying a biological sample from the mouse for immunogenicity.

In some embodiments, the method further comprises obtaining the huPBMCs from a human subject.

In some embodiments, the method further comprises predicting how the human subject will respond to the human therapeutic agent based on the assaying of the biological sample from the mouse.

In some embodiments, the human therapeutic agent is selected from vaccines, antibodies, recombinant protein therapeutics (e.g., growth factors), cell-based therapies (e.g., chimeric antigen receptor (CAR)-T cell, TCR-engineered T cell, tumor-infiltrating lymphocyte (TIL), and regulatory T cell (Treg) therapies), DNA-based (e.g., gene, antisense oligonucleotide) therapies, and RNA-based (e.g., RNAi and mRNA) therapies.

In some embodiments, the assaying comprises characterizing plasma or human B cell function.

In some embodiments, the assaying comprises detecting antigen-specific human T cells and/or activation markers on human B cells, human T cells, and/or human myeloid cells.

Yet other aspects provide a method comprising: administering to the humanized immunodeficient mouse of any one of the preceding paragraphs an agent, such as a chemical agent or protein agent, that induces a human autoimmune response characteristic of a human autoimmune disease; administering a human therapeutic agent to the humanized immunodeficient mouse; and assaying a biological sample from the mouse for an inflammatory response.

In some embodiments, the method further comprises obtaining the huPBMCs from a human subject.

In some embodiments, the method further comprises predicting how the human subject will respond to the human therapeutic agent based on the assessing of the biological sample from the mouse.

In some embodiments, the chemical agent is selected from 2,4,6-Trinitrobenzene sulfonic acid (TNBS) (e.g., in ethanol), dextran sulfate sodium (DSS), and oxazolone.

In some embodiments, the autoimmune disease is selected from systemic lupus erythematosus, inflammatory bowel disease (e.g., ulcerative colitis and Crohn's disease), multiple sclerosis, Type 1 diabetes mellitus, psoriasis, and rheumatoid arthritis. In some embodiments, a chemical agent, such as pristane (a natural saturated terpenoid alkane obtained primarily from shark liver oil; 2,6,10,14-tetramethylpentadecane, C₁₉H₄₀), is administered to a mouse to model systemic lupus erythematosus. In other embodiments, a chemical agent, such as TNBS, is administered to a mouse to model inflammatory bowel disease (e.g., ulcerative colitis and Crohn's disease). In yet other embodiments, myelin or a peptide derived from myelin is administered to a mouse to model multiple sclerosis. In some embodiments, type II collagen is administered to a mouse to model rheumatoid arthritis.

In some embodiments, the assaying comprises recording body condition score, fecal score, and/or body weight over time.

In some embodiments, the assaying comprises euthanizing the mouse and weighing the colon of the mouse and/or measuring the length of the colon of the mouse.

Still other aspects provide a method comprising: administering to the humanized immunodeficient mouse of any one of the preceding paragraphs an agent that facilitates sensitization in the mouse; administering a human therapeutic agent to the humanized immunodeficient mouse; optionally challenging the humanized immunodeficient mouse; and assaying a biological sample from the mouse for an inflammatory response.

In some embodiments, the method further comprises obtaining the huPBMCs from a human subject.

In some embodiments, the method further comprises predicting how the human subject will respond to the human therapeutic agent based on the assaying of the biological sample from the mouse.

In some embodiments, the agent is selected from 2,4-Dinitro-1-fluorobenzene (DNFB), oxazolone, and keyhole limpet hemocyanin (KLH).

In some embodiments, the assaying comprises measuring human cytokine and/or chemokine levels.

Some aspects provide a method comprising: administering a human therapeutic agent to the humanized immunodeficient mouse of any one of the preceding paragraphs; and assessing a biological sample from the mouse for human antibodies that bind specifically to the human therapeutic agent.

In some embodiments, the method further comprises obtaining the huPBMCs from a human subject.

In some embodiments, the method further comprises predicting how the human subject will respond to the human therapeutic agent based on the assessing of the biological sample from the mouse.

In some embodiments, the humanized immunodeficient mouse of any one of the preceding paragraphs has not been subjected to a myeloablative treatment.

In some embodiments, the humanized immunodeficient mouse of any one of the preceding paragraphs has not been subjected to irradiation.

### BRIEF DESCRIPTION OF THE DRAWINGS

FIGS. 1A-1D are graphs of data showing that irradiated NSG-Flt3^{null} (hu-FLT3L) huPBMC-engrafted mice display higher levels of CD14+ monocytes in the blood and lungs compared to NSG-SGM3 (hu-IL-15) huPBMC-engrafted mice.

FIGS. 2A-2D are graphs of data showing that irradiated NSG-Flt3^{null} (hu-FLT3L) huPBMC-engrafted mice support expansion of major immune populations including B cells and myeloid subsets.

FIGS. 3A-3F are graphs of data showing that irradiated NSG-Flt3^{null} (hu-FLT3L) mice can be engrafted with a lower number of PBMC and still display high levels of B cells.

FIGS. 4A-4K are graphs of data showing that irradiated NSG-Flt3^{null} (hu-FLT3L) huPBMC-engrafted mice display high levels of monocyte relevant cytokines.

FIGS. 5A-5F are graphs of data showing that irradiated NSG-Flt3^{null} (hu-FLT3L) huPBMC-engrafted mice humanize faster and have a greater expansion of B cells compared to non-irradiated NSG-Flt3^{null} (hu-FLT3L) huPBMC-engrafted mice (engraftment timeline in irradiated (100 cGy) and non-irradiated mice).

FIG. 6 is a graph of data showing that NSG-Flt3^{null} (hu-FLT3L) mice support huPBMC engraftment with or without irradiation.

FIGS. 7A-7I are graphs of data showing that all three irradiated huPBMC-engrafted NSG® mouse models have functional B cells that retain antibody class switching capabilities, but that only NSG-Flt3^{null} (hu-FLT3L) mice contain function B cells when engrafted with huPBMC without prior irradiation.

FIGS. 8A-8B are graphs of data showing that irradiated NSG-Flt3^{null} (hu-FLT3L) mice engrafted with CD3-depleted huPBMC preserve B cell function even without T cell function.

FIGS. 9A-9C are graphs of data showing the frequency of immune cell populations in irradiated NSG-Flt3^{null} (hu-FLT3L) and NSG-SGM3 (hu-IL-15) mice engrafted with huPBMC, and that hu-FLT3L mice display higher levels of hCD45+ cells in the bone marrow and spleen.

FIGS. 10A-10C are graphs of data showing that irradiated NSG-Flt3^{null} (hu-FLT3L) huPBMC-engrafted mice display high levels of monocyte relevant cytokines in a huPBMC dose-dependent manner.

FIGS. 11A-11B are graphs of data showing that irradiated NSG-Flt3^{null} (hu-FLT3L) mice engrafted with huPBMC produce IgG and IgM in a huPBMC dose-dependent manner.

FIGS. 12A-12B are graphs of data showing that non-irradiated NSG-Flt3^{null} (hu-FLT3L) mice engrafted with huPBMC produce tetanus toxoid antibody following administration with a modified Tdap vaccine.

FIGS. 13A-13B are graphs of data showing that non-irradiated NSG-Flt3^{null} (hu-FLT3L) mice engrafted with huPBMC produce increased numbers of engrafted human cells, including B cells and plasma cells, following administration with a modified Tdap vaccine.

FIG. 14 is a graph of data collected from mice (irradiated NSG-Flt3^{null} (hu-FLT3L) mice in which MHC Class I and II alleles have been knocked out and huPBMCs have been engrafted (designated in the figure as “FLT3xDKO”)) showing the percentage of hCD19 cells that express the activation marker CD38, which is associated with activated B cells and plasmablasts, and the plasma cell marker CD138.

FIG. 15 is a graph of data showing that mice (irradiated NSG-Flt3^{null} (hu-FLT3L) mice in which MHC Class I and II alleles have been knocked out and huPBMCs have been engrafted (designated in the figure as “FLT3xDKO”)) challenged with TDaP produced higher levels of human anti-tetanus antibodies compared to mice who did not receive TDaP vaccine.

FIG. 16 is a graph of data collected from irradiated and non-irradiated NSG-Flt3^{null} (hu-FLT3L) mice showing total human IgG produced 26 days (SD26) post engraftment with huPBMCs.

FIG. 17 is a graph of data collected from irradiated and non-irradiated NSG-Flt3^{null} (hu-FLT3L) mice (designated in the figure as “FLT3L”) showing all human immunoglobulin isotypes detected 26 days (SD26) post engraftment with huPBMCs.

### DETAILED DESCRIPTION

### Mouse Models

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat, and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The first Lab Code appended to a strain symbol identifies and credits the creator of the strain. The Lab Code at the end of a strain symbol indicates the current source for obtaining mice of that strain. Different Lab Codes appended to the same strain symbol distinguish sublines and alert the user that there may be genetic divergence between the different sublines. Lab Codes are assigned from a central registry to assure that each is unique. The registry is maintained at the Institute for Laboratory Animal Research (ILAR) at the National Academy of Sciences, Washington, D.C.

Lab Codes may be obtained electronically at ILAR's web site (nas.edu/cls/ilarhome.nsf). See also Davisson M T, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

A mouse model of disease may be modified to enable the assessment of a disease. Any system (e.g., immune, respiratory, nervous, or circulatory), organ (e.g., blood, heart, blood vessels, spleen, thymus, lymph nodes, or lungs), tissue (e.g., epithelial, connective, muscle, and nervous), or cell type (e.g., lymphocytes or macrophages) may be modified, either independently or in combination, to enable studying disease in the models provided herein.

Three conventional methods used for the production of genome-modified mice (e.g., knockout mice, transgenic mice) include DNA microinjection (Gordon and Ruddle, Science 1981: 214: 1244-124, incorporated herein by reference), embryonic stem cell-mediated gene transfer (Gossler et al., Proc. Natl. Acad. Sci. 1986, 83: 9065-9069, incorporated herein by reference) and retrovirus-mediated gene transfer (Jaenisch, Proc. Natl. Acad. Sci. 1976, 73: 1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a nucleic acid encoding human FcRn and/or a chimeric IgG antibody may be confirmed, for example, using any number of genotyping methods (e.g., sequencing and/or genomic PCR).

New mouse models can also be created by breeding parental lines, as described in the Examples herein. With the variety of available mutant, knockout, knockin, transgenic, Cre-lox, Tet-inducible system, and other mouse strains, multiple mutations and transgenes may be combined to generate new mouse models. Multiple mouse strains may be bred together to generate double, triple, or even quadruple and higher multiple mutant/transgenic mice.

In some embodiments, parental mice are bred to produce F1 mice. A parental mouse may be, for example, homozygous, heterozygous, hemizygous, or homozygous null at a particular allele. An allele is one of two or more alternative forms of a gene that arise by mutation and are found at the same location on a chromosome. Homozygous describes a genotype of two identical alleles at a given locus, heterozygous describes a genotype of two different alleles at a locus, hemizygous describes a genotype consisting of only a single copy of a particular gene in an otherwise diploid organism, and homozygous null refers to an otherwise-diploid organism in which both copies of the gene are missing.

### Immunodeficient Mouse Models

Provided herein, in some embodiments, are immunodeficient mouse models. As is known in the art, immunodeficient mice have impaired or disrupted immune systems, such as specific deficiencies in MHC class I (H2-K1^{tm1Bpe}, H2-D1^{tm1Bpe}; (K^{b}D^{b})^{null}), II (H2-Ab1^{em1Mvw}; IA^{null}) or both, B cell or T cell defects, or defects in both, natural killer (NK) cell defects, myeloid defects (e.g., defects in granulocytes and/or monocytes), macrophage defects, dendritic cell defects, as well as immunodeficiency due to knockdown of genes for cytokines, cytokine receptors, TLR receptors and a variety of transducers and transcription factors of signaling pathways. Immunodeficiency mouse models include the single-gene mutation models such as nude-mice (nu) strains, the severe combined immunodeficiency (scid) strains, non-obese diabetic (NOD) strains, RAG (recombination activating gene) strains with targeted gene deletion and a variety of hybrids originated by crossing doubly and triple mutation mice strains with additional defects in innate and adaptive immunity.

In some embodiments, an immunodeficient mouse comprises an interleukin-2 receptor gamma null (IL-2Rγ^{null}) allele. An IL-2Rγ^{null} allele is a null mutation in the gene encoding the interleukin 2 receptor gamma chain (IL2Rγ, homologous to IL2RG in humans), which blocks natural killer (NK) cell differentiation, thereby removing an obstacle that prevents the efficient engraftment of primary human cells (Cao et al., 1995; Greiner et al., 1998; and Shultz et al., 2005, each of which is incorporated herein by reference). In some embodiments, an immunodeficient mouse is homozygous for an IL-2Rγ^{null} allele.

In some embodiments, an immunodeficient mouse comprises a Prkdc^{scid} allele. The Prkdc^{scid} mutation is a loss-of-function (null) mutation in the mouse homolog of the human PRKDC gene—this mutation essentially eliminates adaptive immunity (see, e.g., Blunt et al., 1995; Greiner, Hesselton, & Shultz, 1998), each of which is incorporated herein by reference). In some embodiments, an immunodeficient mouse is homozygous for a Prkdc^{scid} allele.

In some embodiments, an immunodeficient mouse comprises a Rag1^{null} allele. The Rag1^{null} mutation renders the mice B and T cell deficient. In some embodiments, an immunodeficient mouse is homozygous for a Rag1^{null} allele.

An impaired immune system may be measured by any method known in the art including, but not limited to: production of mature immune cells (e.g., B cells, T cells, dendritic cells, macrophages, natural killer cells), deficient endogenous cytokine signaling, limited resistance to infection, and reduced survival. In some embodiments, an immunodeficient mouse lacks mature mouse T cells, lacks mature mouse B cells, lacks functional natural killer cells, and is deficient in endogenous (e.g., mouse) cytokine signaling. Mature T cells develop in the thymus and are released to other tissues, including blood, spleen, and lymphatic system. Mature B cells express pathogen-specific antibodies on their surface. Functional natural killer cells recognize and kill malignant and virally transformed cells without previously being exposed. Endogenous (e.g., mouse) cytokine signaling is important in maintaining homeostasis and relies on cytokines to regulate immune, nervous, and endocrine system function. Deficient endogenous (e.g., mouse) cytokine signaling means that the level of cytokine signaling is not sufficient to maintain immune system homeostasis compared to an endogenous immune system that is not deficient. Lack of mature cells (e.g., T cells or B cells), functional cells, (e.g., natural killer cells), deficient cytokine signaling, or some combination thereof may be a 10-99%, 5%-95%, 20%-90%, 30%-80%, 40%-70%, or 50%-60% decrease compared to a non-immunodeficient mouse. Lack of mature cells (e.g., T cells or B cells), functional cells (e.g., natural killer cells), deficient cytokine signaling, or some combination thereof may be a 10%, 15%, 20%, 25%, 30%, 35%, 40%, 45%, 50%, 55%, 60%, 65%, 70%, 75%, 80%, 85%, 90%, 95%, or 99% decrease compared to a non-immunodeficient mouse.

Lack of mature cells or functional cells (e.g., T cells, B cells, NK cells) may be assessed by any method known in the art including, but not limited to: flow cytometry; quantitative PCR (qPCR) of T cell markers (e.g., CD3, CD8, CD4, CD25, CD127, CD152), B cells markers (e.g., CD19, IgM, BCAP), and NK cells (e.g., CD224, CD122, NK11, NKp46, Ly49, CD11b, CD49b); immunofluorescence, and ELISA. Deficient cytokine signaling (e.g., mouse cytokine signaling) may be assessed by any method known in the art including, but not limited to: flow cytometry, qPCR of cytokines (e.g., IL-2, IL-7, IL-15, IFNγ, IL-4, IL-5, IL-9, IL-13, IL-25, IL-17A, IL-17F, IL-22, TNFα, IL-12, CCL3, GM-CSF, IL-6, IL-10, TGFβ, IL18, IL-21), immunofluorescence, and ELISA.

An immunodeficient mouse may express any human cytokine or combination of human cytokines that increases the efficacy of the immunodeficient mouse as an animal model (e.g., of human IgG antibody pharmacokinetics or activity, of human IgG antibody production, of human disease). A cytokine is a protein or peptide that modulates the activities of individual cells or tissues (e.g., other human cells, mouse cells). Non-limiting examples of types of human cytokines that may be expressed in a human fibrosis model include: hematopoietic cytokines, lymphokines, monokines, interferons, and chemokines.

In some embodiments, an immunodeficient mouse comprises human immune cells that secrete human serum cytokines (e.g., following immune cell stimulation/activation). Non-limiting examples of human serum cytokines include: EGF, Eotaxin, FGF-2, FLT3, Fractalkine, G-CSF, GM-CSF, GROα, IFNα, IFNγ, IL-10, Il-12p40, IL-12p70, IL-13, IL-15, Il-17A, IL-17E, IL-17F, Il-18, IL-1α, Il-1β, IL-RA, IL-2, IL-22, IL-27, IL-3, IL-4, IL-5, IL-6, IL-7, IL-8, IL-9, IP-10, M-CSF, MCP-1, MCP-3, MDC, MIG, MIP-1α, MIP-10, PDGF-AA, PDGF-AB, RANTES, sCD40L, TGFα, TNFα, TNFβ, and VEGF.

In some embodiments, an immunodeficient mouse described herein expresses 1-20, 2-19, 3-18, 4-17, 5-16, 6-15, 7-14, 8-13, 9-12, or 10-11 human cytokines (e.g., human hematopoietic cytokines). In some embodiments, an immunodeficient mouse described herein expresses 1, 2, 3, 4, 5, 6, 7, 8, 9, 10, 11, 12, 13, 14, 15, 16, 17, 18, 19, 20, or more human cytokines. In some embodiments, a human cytokine expressed in an immunodeficient mouse is involved in human antibody persistence and development.

Non-limiting examples of spontaneous and transgenic immunodeficient mouse models include the following mouse strains:
NOD [Kikutani H et al. Adv Immunol 1992; 51: 285-322; and Anderson M S et al. Ann Rev Immunol 2005; 23: 447-85];
Nude (nu) [Flanagan SP. Genet Res 1966; 8: 295-309; and Nehls M et al. Nature 1994; 372: 103-7];
Scid (scid) [Bosma G C et al. Nature 1983; 301:527-30; Mosier D E et al. Nature 1988; 335: 256-9; and Greiner D L et al. Stem Cells 1998; 16: 166-77];
RAG1 and RAG2 (rag) [Mombaerts P et al. Cell 1992; 68: 869-77; Shinkai U et al. Cell 1992; 68: 855-67];
NOD-scid [Greiner D L et al. 1998; Shultz L D et al. J Immunol 1995; 154: 180-91; Melkus M W et al. Nature Med 2006; 12: 1316-22; and Denton P W et al. PLoS Med 2008; 4(12): e357];
IL2rgnull [DiSanto J P et al. Proc Natl Acad Sci USA 1995; 92: 377-81];
B2mnull [Christianson S W et al. J Immunol 1997; 158: 3578-86];
NSG® mice (NOD-scid IL2rγ^{null}) [Shultz L D et al. Nat Rev Immunol 2007; 7: 118-30; Ito M et al. Blood 2002; 100: 3175-82; Ishikawa I et al. Blood 2005; 106: 1565-73; and Macchiarini F et al. J Exp Med 2005; 202: 1307-11];
NRG mice (NOD.Cg-Rag1^{tm1Mom} Il2rg^{tm1Wjl}/SzJ) [Pearson T et al. Clin Exp Immunol 2008 November; 154(2):270-84]
NOG mice (NOD.cg-Prkdc^{scid}rg^{tltm1Sug}) [Shultz L D et al. Nat Rev Immunol 2007; 7: 118-30];
NCG mice (NOD-PrkdC^{em26Cd52}Il2rg^{em26Cd22}/NjuCrl);
NOD-scid B2mnull [Shultz et al. 2007; Shultz L D et al. Transplantation 2003; 76: 1036-42; Islas-Ohlmayer M A et al. J Virol 2004; 78:13891-900; and Macchiarini et al. 2005];
HLA transgenic mice [Grusby M J et al. Proc Natl Acad Sci USA 1993; 90(9): 3913-7; and Roy C J et al. Infect Immun 2005; 73(4): 2452-60]. See, e.g., Belizario J E The Open Immunology Journal, 2009; 2:79-85;
BRG mice (BALB/cA-Rag2^{null}Il2rγ^{null}) [Goldman J P et al. Br J Haematol. 1998; 103:335-342].

Provided herein, in some embodiments, are immunodeficient mouse models having the non-obese diabetic (NOD) mouse genotype. The NOD mouse (e.g., Jackson Labs Stock #001976, NOD-Shi^{Ltj}) is a polygenic mouse model of autoimmune (e.g., Type 1) diabetes, characterized by hyperglycemia and insulitis, a leukocytic infiltration of the pancreatic islet cells. The NOD mice are hypoinsulinemic and hyperglucagonemic, indicating a selective destruction of pancreatic islet beta cells. The major component of diabetes susceptibility in NOD mice is the unique MHC haplotype. NOD mice also exhibit multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. They also lack hemolytic complement C5. NOD mice also are severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background has a genetic background (“background”) selected from NOD-Cg.-Prkdc^{scid}IL2rg^{tm1wJt}/SzJ (NSG®), a NOD.Cg-Rag1^{tm1Mom} Il2rg^{tm1Wjl}/SzJ (NRG), NOD.Cg-Prkdc^{scid}Il2rg^{tm1Sug}/ShiJic (NOG), and NOD-Prkdc^{em26Cd52}Ilrg^{em26Cd22}/NjuCrl (NCG). Other immunodeficient mouse strains are contemplated herein.

In some embodiments, an immunodeficient mouse model based on the NOD background has an NOD-Cg.-Prkdc^{scid}IL2rg^{tm1wJl}/SzJ (NSG®) genetic background. The NSG® mouse (e.g., Jackson Labs Stock No.: #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG® mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2rg^{tmlWjl} targeted mutation. The Il2rg^{tm1Wjl} mutation is a null mutation in the gene encoding the interleukin 2 receptor gamma chain (IL2Rγ, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse model has an NRG genotype. The NRG mouse (e.g., Jackson Labs Stock #007799) is extremely immunodeficient. This mouse comprises two mutations on the NOD/ShiLtJ genetic background; a targeted knockout mutation in recombination activating gene 1 (Rag1) and a complete null allele of the IL2 receptor common gamma chain (IL2rg^{null}). The extreme immunodeficiency of NRG allows the mice to be humanized by engraftment of human CD34⁺ hematopoietic stem cells (HSC) and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL-2 receptor-γ chain knockout (IL2ryKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG genotype. The NCG mouse (e.g., Charles River Stock #572) was created by sequential CR.ISPR/Cas9 editing of the Prkdc and Il2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

Provided herein, in some embodiments, are immunodeficient mouse models that are deficient in MHC Class I, MHC Class II, or MHC Class I and MHC Class II. A mouse that is deficient in MHC Class I and/or MHC Class II does not express the same level of MHC Class I proteins (e.g., α-microglobulin and β2-microglobulin (B2M)) and/or MHC Class II proteins (e.g., a chain and β chain) or does not have the same level of MHC Class I and/or MHC Class II protein activity as a non-immunodeficient (e.g., MHC Class I/II wild-type) mouse. In some embodiments, the expression or activity of MHC Class I and/or MHC Class II proteins is reduced (e.g., by at least 30%, at least 40%, at least 50%, at least 60%, at least 70%, at least 80%, at least 90% or more), relative to a non-immunodeficient mouse.

Immunodeficient mice that are deficient in MHC Class I, MHC Class II, and MHC Class I and MHC Class II are described in International Publication No. WO 2018/209344. In some embodiments, the genetic background of a mouse is NOD.Cg-Prkdc^{scid} H2-K1^{tm1Bpe} H2-Ab1^{em1Mvw} H2-D1tmBpe Il2rg^{tm1Wjl}/SzJ (i.e., NSG-(K^{b} D^{b})^{null} (IA^{null})) In some embodiments, an NSG-(K^{b}D^{b})^{null} (IA^{null}) mouse expresses human FLT3L (e.g., the genome of the mouse comprises a nucleic acid encoding human FLT3L, such as a transgene encoding human FLT3L) and comprises a Flt3^{null} allele (does not express mouse FLT3). In some embodiments, such a mouse is engrafted with human PBMCs.

An NSG-SGM3 mouse is the NSG® derivative mouse NOD.Cg-Prkdc^{scid} Il2rg^{tm1Wjl} Tg(CMV-IL3,CSF2,KITLG)1Eav/MloySzJ (Jackson Laboratory Stock No: 013062). The transgenic NSG-SGM3 mice express three human cytokines: human Interleukin-3 (IL-3), human Granulocyte/Macrophage-colony stimulating factor 2 (GM-CSF), and human Stem Cell Factor (SCF). NSG-SGM3 mice combine the features of the highly immunodeficient NSG® mouse with expression of human cytokines IL-3, GM-CSF, and SCF that support stable engraftment of myeloid lineages (e.g., monocytes, dendritic cells) and regulatory T cell populations.

In some embodiments, an NSG® mouse transgenically expresses human IL-15. An NSG-IL-15 mouse, NOD.Cg-Prkdc^{scid} Il2rg^{tm1wWjl} Ig(IL-15)1Sz/SzJ (Jackson Laboratory Stock No: 030890), expresses human IL-15 and is combined with the highly immunodeficient NOD scid gamma (NSG®) mouse. Expression of human IL-15, in some embodiments, enhances the development of human NK cells in immunodeficient mice engrafted with human stem cells.

In some embodiments, an NSG-SGM3 mouse transgenically expresses human IL-15 (i.e., NSG-SGM3 (hu-IL-15)). In some embodiments, such a mouse is engrafted with human PBMCs.

### Inactivated Flt3 Allele

In some embodiments, an immunodeficient mouse comprises an inactivated Flt3 allele. Flt3 encodes FMS-like tyrosine kinase 3 (FLT3), which enables several functions, including phosphatidylinositol 3-kinase binding activity, protein tyrosine kinase activity, and ubiquitin protein ligase binding activity. FLT3 is also involved in several processes, including common myeloid progenitor cell proliferation, hemopoiesis, and lymphocyte proliferation. Some aspects provide an immunodeficient mouse that comprises an inactivated Flt3 allele. In some embodiments, an immunodeficient mouse comprises an Flt3^{null} allele. In some embodiments, an immunodeficient mouse is homozygous for an Flt3^{null} allele. Thus, in some embodiments, an immunodeficient mouse does not express a detectable level of mouse FLT3. A detectable level of mouse FLT3 is any level of FLT3 protein detected using a standard protein detection assay, such as flow cytometry and/or an ELISA. In some embodiments, an immunodeficient mouse expresses an undetectable level or a low level of mouse FLT3. For example, a mouse may express less than 1,000 μg/ml mouse FLT3. In some embodiments, an immunodeficient mouse expresses less than 500 μg/ml mouse FLT3 or less than 100 μg/ml mouse FLT3. The mouse FLT3 receptor is also referred to as cluster of differentiation antigen CD135. Thus, in some embodiments, an immunodeficient mouse does not comprise (there is an absence of) CD135+ multipotent progenitor (MPP3) cells. A nonlimiting example of an immunodeficient mouse comprising an inactivated Flt3 allele is described in International Publication No. WO 2020/168029.

### Human FLT3L Transgene

Some aspects provide an immunodeficient mouse that comprises a human FLT3L transgene (e.g., integrated into the genome of the mouse). Human FLT3L encodes FMS-related receptor tyrosine kinase 3 ligand (FLT3L), which controls the development of dendritic cells (DCs) and is particularly important for plasmacytoid DCs and CD8-positive classical DCs and their CD103-positive tissue counterparts. In some embodiments, an immunodeficient mouse expresses human FLT3L protein. In some embodiments, human FLT3L is expressed at a level of at least 5,000 pg/ml or at least 10,000 pg/ml. For example, human FLT3L may be expressed at a level of at least 5,000 pg/ml, 7,500 pg/ml, 10,000 pg/ml, 12,500 pg/ml, 15,000 pg/ml, 17,500 pg/ml, 20,000 pg/ml, 22,500 pg/ml, 25,000 pg/ml, 27,500 pg/ml, 30,000 pg/ml, 32,500 pg/ml, 35,000 pg/ml, 37,500 pg/ml, 40,000 pg/ml, 42,500 pg/ml, 45,000 pg/ml, 47,500 pg/ml, or 50,000 pg/ml. In some embodiments, human FLT3L is expressed at a level of 10,000 pg/ml to 30,000 pg/ml. In some embodiments, human FLT3L is expressed at a level of 15,000+/−1000 pg/mL to 17,000+/−100 pg/ml. Methods of detecting FLT3L protein expression are known and may be used as provided herein. For example, flow cytometry and/or an ELISA (enzyme-linked immunosorbent assay) using an anti-FLT3L antibody may be used to detect the level of human FLT3L protein present in mouse tissue and/or blood. A nonlimiting example of an immunodeficient mouse comprising a human FLT3L transgene is described in International Publication No. WO 2020/168029.

In some embodiments, an immunodeficient mouse may also comprise an endogenous Flt3L allele. Thus, in some embodiments, an immunodeficient mouse may also express endogenous mouse FLT3L protein. In some embodiments, mouse FLT3L is expressed at a level of at least 1,000 μg/ml or at least 2,000 μg/ml. For example, mouse FLT3L may be expressed at a level of 3,000 μg/ml, 4,000 μg/ml, 5,000 μg/ml, 6,000 μg/ml, 7,000 μg/ml, 8,000 μg/ml, 9,000 μg/ml, or 10,000 μg/ml. In some embodiments, mouse FLT3L is expressed at a level of 5,000 μg/ml to 10,000 μg/ml. In some embodiments, mouse FLT3L is expressed at a level of 6,000 μg/ml to 8,000 ml.

### Humanized Mouse Models Supporting Adaptive Immunity

Surprisingly, as discussed above, the humanized mouse models provided herein support expansion, development, and maturation of cells of the human adaptive immune system and cells of the human innate immune system, including functional human B cells as well as human monocytes, T cells, NK cells, and all three subsets of dendritic cells (i.e., cDC₁, cDC₂, and plasmacytoid DC cells).

Immunodeficient mice engrafted with functional human cells and/or tissues are referred to as “humanized mice.” As used herein, the terms “humanized mouse”, “humanized immune deficient mouse”, “humanized immunodeficient mouse”, and the plural versions thereof are used interchangeably to refer to an immunodeficient mouse humanized by engraftment with functional human cells and/or tissues, such as human peripheral blood mononuclear cells (PBMCs), as described below.

Humanized mice are generated by starting with an immunodeficient mouse (e.g., an immunodeficient mouse of 2, 3, 4, 5, 6, 7, 8, 9, 10, or more weeks of age) and, if necessary, depleting and/or suppressing any remaining murine immune cells (e.g., chemically or with radiation, i.e., myeloablation). That is, successful survival of the human immune system in the immunodeficient mice may require suppression of the mouse's immune system to prevent GVHD (graft-versus-host disease) rejections. After the immunodeficient mouse's immune system has been sufficiently suppressed, the mouse is engrafted with human cells (e.g., PBMCs). As used herein, “engraft” refers to the process of the human cells migrating to, and incorporating into, an existing tissue of interest in vivo. With respect to a humanized immunodeficient mouse, the engrafted human cells repopulate the mouse with a functional human immune system.

The engrafted human cells (e.g., PBMCs) for humanization, in some embodiments, are human leukocyte-antigen (HLA)-matched to the human cells (e.g., human cancer cells) of the mouse models. HLA-matched refers to cells that express the same major histocompatibility complex (MHC) genes. Engrafting mice with HLA-matched human xenografts and human immune cells, for example, reduces or prevents immunogenicity of the human immune cells. In some embodiments, a humanized mouse provided in the present disclosure is engrafted with human PBMCs that are HLA-matched to a patient-derived xenograft (PDX) or human cancer cell line.

The engrafted human cells (e.g., PBMCs) for humanization, in some embodiments, are not HLA-matched to the human cells (e.g., human cancer cells) of the mouse models. That is, in some embodiments, a humanized mouse provided in the present disclosure is engrafted with human PBMCs that are not HLA-matched to a PDX or human cancer cell line.

### Myeloablation

In some embodiments, immunodeficient mice are treated to deplete and/or suppress any remaining murine immune cells (e.g., chemically and/or with radiation). In some embodiments, immunodeficient mice are treated only chemically or only with radiation. In other embodiments, immunodeficient mice are treated both chemically and with radiation.

In some embodiments, immunodeficient mice are administered a myeloablative agent, that is, a chemical agent that suppresses or depletes murine immune cells. Examples of myeloablative agents include busulfan, treosulfan, dimethyl mileran, melphalan, and thiotepa.

In some embodiments, immunodeficient mice are irradiated prior to engraftment with human cells, such as human HSCs and/or PBMCs. It is thought that irradiation of an immunodeficient mouse destroys mouse immune cells in peripheral blood, spleen, and bone marrow, which facilitates engraftment of human cells, such as human PBMCs (e.g., by increasing human cell survival factors), as well as expansion of other immune cells. Irradiation also shortens the time it takes to accumulate the required number of human immune cells to “humanize” the mouse models.

For immunodeficient mice (e.g., NSG® mice), this preparation is commonly accomplished through whole-body gamma irradiation. Irradiators may vary in size depending on their intended use. Animals are generally irradiated for short periods of time (less than 15 min). The amount of time spent inside the irradiator varies depending on the radioisotope decay charts, amount of irradiation needed, and source of ionizing energy (that is, X-rays versus gamma rays, for which a cesium or cobalt source is needed).

A myeloablative irradiation dose is usually 700 to 1300 cGy, though in some embodiments, lower doses such as 1-100 cGy (e.g., about 2, 5, or 10 cGy), or 300-700 cGy may be used.

As an example, the mouse may be irradiated with 100 cGy X-ray (or 75 cGy-125 cGy X-ray). In some embodiments, the dose is about 1, 2, 3, 4, 5, 10, 20, 100, 125, 150, 175, 200, 300, 400, 500, 600, 700, 800, 900, 1000, 1100, 1200, or 1300 cGy, or between any of the two recited doses herein, such as 100-300 cGy, 200-500 cGy, 600-1000 cGy, or 700-1300 cGy.

### Engraftment of Peripheral Blood Mononuclear Cells

In some embodiments, the immunodeficient mice are engrafted with PBMCs, thereby humanizing the mice. Engraftment refers to the process of the human cells migrating to, and incorporating into, an existing tissue of interest in vivo. The PBMCs may be engrafted after irradiation and before engraftment of human diseased cells (e.g., human cancer cells), after irradiation and concurrently with engraftment of human diseased cells, or after irradiation and after engraftment of human diseased cells.

Peripheral blood mononuclear cells (PBMCs) are peripheral blood cells having a round nucleus. These mononuclear blood cells recirculate between tissues and blood and are a critical component in the immune system to fight infection and adapt to intruders. There are two main types of PBMCs: lymphocytes and monocytes. The majority (˜70-90%) of an enriched human PBMC sample is composed of lymphocytes (white blood cells), which include CD4+ helper T cells, CD8+ killer T cells, B cells, and Natural Killer (NK) cells. Monocytes make up a smaller portion (˜10-30%) of the enriched human PBMC sample. Monocytes, when stimulated, can differentiate into macrophages or dendritic cells.

PBMCs may be isolated from whole blood samples, for example (e.g., Ficoll gradient). In some embodiments, PBMCs from a subject (e.g., a human subject) with a current or previous diagnosis of cancer or an autoimmune disease may be used. In some embodiments, PBMCs from a subject (e.g., a human subject) with a current or previous diagnosis of cancer or an autoimmune disease may be used.

Methods of engrafting immunodeficient mice with PBMCs to yield a humanized mouse model include but are not limited to intraperitoneal or intravenous injection (Shultz et al., J Immunol, 2015, 174:6477-6489; Pearson et al., Curr Protoc Immunol. 2008; 15-21; Kim et al., AIDS Res Hum Retrovirus, 2016, 32(2): 194-2020; Yaguchi et al., Cell & Mol Immunol, 2018, 15:953-962).

In some embodiments, an immunodeficient mouse is engrafted with 0.5×10⁶-50×10⁶ human PBMCs. For example, an immunodeficient mouse may be engrafted with 0.5×10⁶-50×10⁶, 1×10⁶-50×10⁶, 2×10⁶-50×10⁶, 5×10⁶-50×10⁶, 10×10⁶-50×10⁶, 15×10⁶-50×10⁶, 20×10⁶-50×10⁶, 0.5×10⁶-20×10⁶, 1×10⁶-20×10⁶, 2×10⁶-20×10⁶, 5×10⁶-20×10⁶, 10×10⁶-20×10⁶, 15×10⁶-20×10⁶, 0.5×10⁶-15×10⁶, 1×10⁶-15×10⁶, 2×10⁶-15×10⁶, 5×10⁶-15×10⁶, 10×10⁶-15×10⁶, 0.5×10⁶-10×10⁶, 1×10⁶-10×10⁶, 2×10⁶-10×10⁶, 5×10⁶-10×10⁶, 0.5×10⁶-5×10⁶, 1×10⁶-5×10⁶, 2×10⁶-10×10⁶, 0.5×10⁶-2×10⁶, 1×10⁶-2×10⁶, or 0.5×10⁶-1×10⁶ human PBMCs. In some embodiments, an immunodeficient mouse is engrafted with about 0.5×10⁶, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, about 9.5×10⁶, or about 10×10⁶ human PBMCs.

In some embodiments, an immunodeficient mouse is engrafted with human PBMCs, for example, following a myeloablative treatment, such as sublethal irradiation. In some embodiments, an immunodeficient mouse is engrafted with human PBMCs about 15 minutes, 30 minutes, 45 minutes, 1 hour, or more following the myeloablative treatment. In some embodiments, an immunodeficient mouse is engrafted with human PBMCs about 1 to 5 days, about 1 to 10 days, or about 1 to 20 days, e.g., about 1, 2, 3, 4, 5, 6, 7, 8, 9, 10, 11, 12, 13, 14, 15, 16, 17, 18, 19, or 20 days following the myeloablative treatment.

### Human Immune Cells

The mouse models provided herein support expansion, development, and maturation of a complete repertoire of human immune cells, including cells of the innate immune system and cells of the adaptive immune system. There are two main lineages of human immune cells: the lymphoid lineage and the myeloid lineage. Progenitor cells of the lymphoid lineage develop into B cell progenitors, natural killer cells, and T cell progenitors. B cell progenitors continue to develop into either memory B cells or plasma cells, while T cell progenitors continue to develop into memory T cells, cytotoxic T cells, or helper T cells. Progenitor cells of myeloid lineage develop into neutrophils, eosinophils, basophils, mast cells, and monocytes. The monocytes further develop into dendritic cells (e.g., cDC1s or cDC2s) and macrophages.

The human immune system includes the innate immune system and the adaptive immune system. The innate immune system includes the mononuclear phagocyte system of macrophages, dendritic cells, and monocytes, natural killer cells, mast cells, γδ T cells, natural killer T cells, and granulocytes (basophils, eosinophils, and neutrophils). The adaptive immune system includes humoral immunity (also referred to as B cell immunity), which includes B cells that produce antibodies, and cellular immunity (also referred to as T cell immunity), which includes CD4⁺ and CD8⁺ T cells, natural killer cells, and γδ T cells.

T cells are key actors of the adaptive immune system, are commonly identified by CD3 expression, and detect antigen through T cell receptors (TCRs), which recognize peptides presented by the major histocompatibility complex (MHC). Circulating tumor cell antigens are delivered to lymph nodes, where they are displayed to CD4⁺ and CD8⁺ T cells, also known as T helper and cytotoxic T cells, respectively. Following activation. T belper cells release a variety of cytokines, including IFNγ. Cytotoxic T cells recognize cells expressing tumor-specific antigens and kill them through perforin-or granzyme-induced apoptosis.

Expression of a variety of molecules is used to indicate T cell function. Both CD69 and CD25 are upregulated via TCR signaling but with distinctive kinetics, with CD69 detectable within hours of TCR ligation and CD25 increasing later. T cell exhaustion, defined by poor effector function and which arises during chronic infections and cancer, is characterized by expression of PD-1, TIM-3, and LAG3; however, these molecules are also upregulated during T cell activation. Other types of T cells—including naïve, memory, and effector—are distinguished from one another via a combination of CD45RA. CD45RO, and CD62L or CCR7. Multiple subtypes of CD4⁺ T cells that secrete different cytokines—and induce different immune responses—can be identified by unique expression of transcription factors. For example, T-Bet, typically expressed by Th1 cells, generally denotes an antitumor phenotype and IFNγ production. FoxP3, expressed by regulatory T cells (Treg), signifies a protumor phenotype that suppresses the antitumor immune response through cytokine production and other mechanisms.

Dendritic cells (DCs) are part of the innate immune system and play a key role in initiating adaptive immunity via both antigen presentation to activate naïve T cells and cytokine secretion. DCs are broadly divided into plasmacytoid and conventional subclasses. Plasmacytoid DCs are identified by co-expression of Siglec-H and CD317 and specialize in producing large amounts of type I IFNγ, whereas conventional DCs are characterized by co-expression of CD11c and HLA-DR and specialize in antigen presentation to T cells. Conventional DCs are further subdivided into those that express CDIc and facilitate activation of CD4⁺ T cells and those that express CD141, XCR1, or CLEC9A and activate CD8⁺ T cells by cross-presentation.

Macrophages are also cells of the innate immune system and are identified by expression of CD68 and MHCII and lack of CD11c. They specialize in phagocytosis and also secrete cytokines that influence the immune response. Macrophages generally are classified as pro-inflammatory (M1-like) or anti-inflammatory (M2-like). M1-like macrophages are identified by expression of CD80, CD86, or iNOS and promote the antitumor immune response by phagocytosis of malignant cells and production of T cell-activating ligands. Conversely, M2-like macrophages are identified by expression of CD163 or CD206 and can promote tumor growth through secretion of immunosuppressive cytokines, such as IL-10, and by promoting a Th2 response. M2 macrophages can also express the immunosuppressive enzyme arginase, which depletes arginine from the tumor microenvironment, leading to reduced T cell proliferation and function.

Natural killer (NK) cells represent the primary innate immune cell type. They recognize and kill cancer by detecting downregulation of MHC class I on tumor cells and/or by detecting upregulation of ligands on tumor cells that bind to activating receptors on NK cells. NK cells are commonly identified by a combination of CD56 and CD16 and lack of CD3 expression.

Myeloid-derived suppressor cells (MDSCs) are a diverse population of immature immunosuppressive cells that are present in a variety of tumors. They have been shown to inhibit CD8⁺ T cell activation through the expression of NOS2 and arginase 1, induce Treg development, and polarize macrophages to an M2-like phenotype. MDSCs consist of 2 large groups of cells, termed monocytic or polymorphonuclear. Several open questions remain regarding these immunosuppressive cells, including whether they are truly distinct from neutrophils and monocytes, the mechanisms regulating their accumulation and differentiation, and how they contribute to resistance to anticancer therapies. The specific markers of MDSCs remain under active investigation. Currently, they are most often identified by expression of CD11b, lack of HLA-DR expression, and expression of either CD14 for monocytic MDSCs or CD15 for polymorphonuclear MDSCs.

Some aspects of the present disclosure provide mouse models that support expansion, development, and/or maturation of cells of the adaptive immune system and cells of the innate immune system. In some embodiments, a mouse model supports expansion, development, and/or maturation of human myeloid cells and human lymphoid cells. In some embodiments, a mouse model supports expansion, development, and/or maturation of human CD45⁺ immune cells. In some embodiments, a mouse model supports expansion, development, and/or maturation of human CD19⁺ B cells (e.g., human B cells that produce circulating immunoglobulin (Ig)). In some embodiments, a mouse model supports expansion, development, and/or maturation of human CD14⁺ monocytes. In some embodiments, a mouse model supports expansion, development, and/or maturation of human CD3⁺ T cells. In some embodiments, a mouse model supports expansion, development, and/or maturation of human CD11c⁺CD141⁺ dendritic cells (cDC1s cells). In some embodiments, a mouse model supports expansion, development, and/or maturation of human CD11c⁺CD1c⁺ dendritic cells (cDC2s cells). In some embodiments, a mouse model supports expansion, development, and/or maturation of human CD11c⁻CD123⁺ plasmacytoid dendritic cells.

### B Cell Antibody Class Switching

The mouse models provided herein, in some embodiments, unexpectedly support expansion of mature B cells that are capable of immunoglobulin (Ig) class switching, which is a biological mechanism that changes a B cell's production of Ig from one type to another, such as from the isotype IgM to the isotype IgG. Class switching occurs after activation of a mature B cell via its membrane-bound antibody molecule (or B cell receptor) to generate the different classes of antibody, all with the same variable domains as the original antibody generated in the immature B cell during the process of V(D)J recombination, but possessing distinct constant domains in their heavy chains.

Naïve mature B cells produce both IgM and IgD, which are the first two heavy chain segments in the immunoglobulin locus. After activation by antigen, these B cells proliferate. If these activated B cells encounter specific signaling molecules via their CD40 and cytokine receptors (both modulated by T helper cells), they undergo antibody class switching to produce IgG, IgA or IgE antibodies. During class switching, the constant region of the immunoglobulin heavy chain changes but the variable regions do not, and therefore antigenic specificity, remains the same. This allows different daughter cells from the same activated B cell to produce antibodies of different isotypes or subtypes (e.g., IgG1, IgG2 etc.).

### Nucleic Acids: Engineering and Delivery

A mouse described herein comprises a nucleic acid encoding a human FLT3L. In some embodiments, the mouse comprises a transgene encoding human FLT3L, integrated into the genome of the mouse.

The nucleic acids provided herein, in some embodiments, are engineered. An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally-occurring nucleic acid). A genomic modification is thus any manipulation of a nucleic acid in a genome (e.g., in a coding region, non-coding region, and/or regulatory region), relative to the corresponding wild-type nucleic acid (e.g., the naturally-occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A loss-of-function mutation, as is known in the art, results in a gene product with little or no function. A null mutation, which is a type of loss-of-function mutation, results in a gene product with no function. In some embodiments, an inactivated allele is a null allele. Other examples of loss-of-function mutations includes missense mutations and frameshift mutations.

A nucleic acid, such as an allele or alleles of a gene, may be modified such that it does not produce a detectable level of a functional gene product (e.g., a functional protein). Thus, an inactivated allele is an allele that does not produce a detectable level of a functional gene product (e.g., a functional protein). A detectable level of a protein is any level of protein detected using a standard protein detection assay, such as flow cytometry and/or an ELISA. In some embodiments, an inactivated allele is not transcribed. In some embodiments, an inactivated allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example.

Methods for delivering nucleic acids to mouse embryos (e.g., mouse) for the production of transgenic mice include, but are not limited to, electroporation (see, e.g., Wang W et al. J Genet Genomics 2016; 43(5):319-27; WO 2016/054032; and WO 2017/124086, each of which is incorporated herein by reference), DNA microinjection (see, e.g., Gordon and Ruddle, Science 1981: 214: 1244-124, incorporated herein by reference), embryonic stem cell-mediated gene transfer (see, e.g., Gossler et al., Proc. Natl. Acad. Sci. 1986; 83: 9065-9069, incorporated herein by reference), and retrovirus-mediated gene transfer (see, e.g., Jaenisch, Proc. Natl. Acad. Sci. 1976; 73: 1260-1264, incorporated herein by reference), any of which may be used as provided herein.

### Genomic Editing Methods

The present application contemplates the use of a variety of gene editing technologies using engineered nucleic acids, for example, to knockout a target gene (e.g., Flt3) or to introduce nucleic acids into the genome of a mouse (e.g., to produce a transgenic mouse). An immunodeficient Flt3^{null} mouse may be produced by any gene editing technology known in the art. In some embodiments, an immunodeficient Flt3^{null} mouse further comprises a human FLT3L transgene (integrated into its genome).

Engineered nucleic acids, such as guide RNAs, donor polynucleotides, and other nucleic acid coding sequences, for example, may be introduced to a genome of an embryo or cell (e.g., stem cell) using any suitable method. The present application contemplates the use of a variety of gene editing technologies, for example, to delete nucleic acids from the genome of an embryo or cell to produce a knockout mouse or to introduce nucleic acids into the genome of an embryo or cell to produce a transgenic mouse. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405.

In some embodiments, a CRISPR system is used to edit the genome of mouse (e.g., mouse) embryos provided herein. See, e.g., Harms D W et al., Curr Protoc Hum Genet. 2014; 83: 15.7.1-15.7.27; and Inui M et al., Sci Rep. 2014; 4: 5396). For example, Cas9 mRNA or protein, one or multiple guide RNAs (gRNAs), and/or a donor nucleic acid can be delivered, e.g., injected or electroporated, directly into mouse embryos at the one-cell (zygote) stage or a later stage to facilitate homology directed repair (HDR), for example, to delete a nucleic acid sequence from the genome or to introduce an engineered nucleic acid (e.g., donor nucleic acid) into the genome.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337: 816-821 and Deltcheva et al., Nature, 2011; 471: 602-607.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

A donor nucleic acid typically includes a sequence of interest flanked by homology arms. Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

### Anti-Drug Antibody Assessment

Some aspects provide method of using the mouse models described herein to assess anti-drug antibodies (ADA) with human therapeutics. ADA may induce unwanted side effects, especially in biotechnology-derived pharmaceuticals, such as therapeutic antibodies and growth factors. Thus, ADA have been subjected to increasing scrutiny by the regulatory authorities using immunogenicity safety studies. ADA have been observed in preclinical and clinical studies resulting in significant changes in toxicology, pharmacokinetics, and efficacy. These effects result from the generation of drug-induced (neutralizing) autoantibodies against, for example, erythropoietin (EPO) Factor VIII (FVIII), or insulin and can be responsible for allergic reactions, or even anaphylactic shock. As a consequence, studies on ADA have become inevitable for bioengineered pharmaceuticals including biosimilars. Adverse immunological reactions may vary widely, depending on how the active ingredients are structured, produced and applied. For example, the expression of anti-Fc antibodies, anti-idiotypic antibodies or antibodies against glycosylated antigens may appear. The detection and characterization assays for ADA must therefore be developed, customized and optimized for each drug.

The humanized immunodeficient mouse models provided herein, in some aspects, are used as in vivo models to assess ADA, for example, long term development of ADA.

In some embodiments, a mouse model used to assess ADA undergoes a myeloablative procedure, such as irradiation or chemical ablation.

In some embodiments, a mouse model used to assess ADA is engrafted with huPBMCs (e.g., target drug naïve huPBMC). In some embodiments, an immunodeficient mouse is engrafted with 0.5×10⁶-50×10⁶ human PBMCs. For example, an immunodeficient mouse may be engrafted with 0.5×10⁶-50×10⁶, 1×10⁶-50×10⁶, 2×10⁶-50×10⁶, 5×10⁶-50×10⁶, 10×10⁶-50×10⁶, 15×10⁶-50×10⁶, 20×10⁶-50×10⁶, 0.5×10⁶-20×10⁶, 1×10⁶-20×10⁶, 2×10⁶-20×10⁶, 5×10⁶-20×10⁶, 10×10⁶-20×10⁶, 15×10⁶-20×10⁶, 0.5×10⁶-15×10⁶, 1×10⁶-15×10⁶, 2×10⁶-15×10⁶, 5×10⁶-15×10⁶, 10×10⁶-15×10⁶, 0.5×10⁶-10×10⁶, 1×10⁶-10×10⁶, 2×10⁶-10×10⁶, 5×10⁶-10×10⁶, 0.5×10⁶-5×10⁶, 1×10⁶-5×10⁶, 2×10⁶-10×10⁶, 0.5×10⁶-2×10⁶, 1×10⁶-2×10⁶, or 0.5×10⁶-1×10⁶ human PBMCs. In some embodiments, an immunodeficient mouse is engrafted with about 0.5×10⁶, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, about 9.5×10⁶, or about 10×10⁶ human PBMCs.

Following engraftment, a target drug may then be administered. The term “target drug” encompasses human therapeutic modalities that elicit a humoral response. Non-limiting examples include therapeutic antibodies, recombinant protein therapeutics (e.g., growth factors), cell-based therapies (e.g., chimeric antigen receptor (CAR)-T cell, TCR-engineered T cell, tumor-infiltrating lymphocyte (TIL), and regulatory T cell (Treg) therapies), DNA-based (e.g., gene, antisense oligonucleotide) therapies, and RNA-based (e.g., RNAi and mRNA) therapies.

In some embodiments, a target drug is administered to the mouse about 4 to 10 days following administration of the huPBMCs. For example, a target drug may be administered 4, 5, 6, 7, 8, 9, or 10 days following administration of the huPBMCs. In some embodiments, a target drug is administered to the mouse at least 4 days, at least 5 days, at least 6 days, at least 7 days, at least 8 days, at least 9 days, or at least 10 days following administration of the huPBMCs. In some embodiments, a single dose of the target drug is administered. In other embodiments, multiple doses (e.g., 2-10, e.g., 2, 3, 4, 5, 6, 7, 8, 9, or 10) doses of the target drug are administered (e.g., weekly, every other week, every three weeks, or monthly). In some embodiments, a target drug is administered according to a standardized dosing schedule.

Anti-drug antibodies may be assessed over a period of time, for example, about 7 to about 270 days. In some embodiments, ADA are assessed by collecting a blood sample from the mouse and characterizing the plasma and/or B cell function. In some embodiments, flow cytometry or ELISA assays are used to assess the blood sample (or other biological sample) for antibody recognition and/or neutralization of the target drug. An initial bleed, prior to target drug administration, may serve as a control, for example. In some embodiments, anti-drug Ig levels increase over time.

An analysis of ADA may include a characterization of ADA titer, neutralizing capacity, binding affinity, isotyping, and other characteristics. There are several isotypes of ADA. For example, IgM ADA may be an early marker of ADA formation, and the presence of IgE antibodies may indicate an allergic reaction against the target drug. In addition, the measurement of IgG subclasses may be supportive for the biological activities of ADA since in humans, IgG1 and IgG3 are mainly involved in complement activation and are more prone to NK cell recognition. The measurement of binding affinities of ADA is also informative for the ADA response interpretation.

### Immunogenicity

Some aspects provide method of using the mouse models described herein to assess immunogenicity of human therapeutic agent, such as antibodies, recombinant protein therapeutics (e.g., growth factors), cell-based therapies (e.g., chimeric antigen receptor (CAR)-T cell, TCR-engineered T cell, tumor-infiltrating lymphocyte (TIL), and regulatory T cell (Treg) therapies), DNA-based (e.g., gene, antisense oligonucleotide) therapies, and RNA-based (e.g., RNAi and mRNA) therapies. Immunogenicity is a measure of the ability of the above modalities to produce an immune response and guides how long that response will last over time. Immunogenicity studies typically work out what type of immune response a vaccine or disease will trigger and how long it will last. In some embodiments, a mouse model described herein is used to assess how suitable a vaccine will be, or is, against different types of viruses, for example. In some embodiments, a mouse model described herein is used to aid in the determination of a correct dose of a vaccine and/or if and/or when a booster vaccination(s) may be required.

The humanized immunodeficient mouse models provided herein, in some aspects, are used as in vivo models to assess immunogenicity, for example, of a therapeutic vaccine or a prophylactic vaccines.

In some embodiments, a mouse model used to assess immunogenicity undergoes a myeloablative procedure, such as irradiation or chemical ablation.

In some embodiments, a mouse model used to assess immunogenicity is engrafted with huPBMCs (e.g., target drug naïve huPBMC). In some embodiments, an immunodeficient mouse is engrafted with 0.5×10⁶-50×10⁶ human PBMCs. For example, an immunodeficient mouse may be engrafted with 0.5×10⁶-50×10⁶, 1×10⁶-50×10⁶, 2×10⁶-50×10⁶, 5×10⁶-50×10⁶, 10×10⁶-50×10⁶, 15×10⁶-50×10⁶, 20×10⁶-50×10⁶, 0.5×10⁶-20×10⁶, 1×10⁶-20×10⁶, 2×10⁶-20×10⁶, 5×10⁶-20×10⁶, 10×10⁶-20×10⁶, 15×10⁶-20×10⁶, 0.5×10⁶-15×10⁶, 1×10⁶-15×10⁶, 2×10⁶-15×10⁶, 5×10⁶-15×10⁶, 10×10⁶-15×10⁶, 0.5×10⁶-10×10⁶, 1×10⁶-10×10⁶, 2×10⁶-10×10⁶, 5×10⁶-10×10⁶, 0.5×10⁶-5×10⁶, 1×10⁶-5×10⁶, 2×10⁶-10×10⁶, 0.5×10⁶-2×10⁶, 1×10⁶-2×10⁶, or 0.5×10⁶-1×10⁶ human PBMCs. In some embodiments, an immunodeficient mouse is engrafted with about 0.5×10⁶, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, about 9.5×10⁶, or about 10×10⁶ human PBMCs.

Following engraftment, a vaccine may then be administered. Non-limiting examples of vaccines include: live vaccines, attenuated vaccines; killed or inactivated vaccines; toxoids; subunit or conjugate vaccines; mRNA vaccines; and viral vector vaccines.

In some embodiments, a vaccine is administered to the mouse about 4 to 10 days following administration of the huPBMCs. For example, a vaccine may be administered 4, 5, 6, 7, 8, 9, or 10 days following administration of the huPBMCs. In some embodiments, a vaccine is administered to the mouse at least 4 days, at least 5 days, at least 6 days, at least 7 days, at least 8 days, at least 9 days, or at least 10 days following administration of the huPBMCs. In some embodiments, a vaccine is administered according to a standardized dosing schedule.

Immunogenicity may be assessed over a period of time, for example, about 7 to about 270 days. In some embodiments, immunogenicity is assessed by collecting a blood sample (or other biological sample, such as a tissue sample (e.g., spleen tissue)) from the mouse and characterizing the plasma and/or B cell function. In some embodiments, flow cytometry is used to assess the blood sample (or other biological sample). Immunogenicity may be assessed, for example, using assays for the detection of vaccine antigen antibodies (e.g., antibodies that specifically bind to a viral protein/antigen), assays for the detection of antigen-specific T cells, and/or assays for activation markers on B cells, T cells, and/or myeloid cells. An initial bleed, prior to vaccine administration, may serve as a control, for example.

### Autoimmunity

Some aspects provide method of using the mice described herein to model and assess human autoimmunity. Functional, mature B cells are critical for promoting autoimmunity. B cells are major effector cells in autoimmunity through antibody production, T cell help and pro-inflammatory cytokine production. B cells were primary identified for their key role as enhancers of the immune response in autoimmunity because they give rise to autoantibody producing plasma cells and promote CD4+ T cell responses by antigen presentation. The B cells bearing these functions are usually considered as effector B cells. The mouse models provided herein support expansion, development, and maturation of functional human B cells as well as human monocytes, T cells, NK cells, and all three subsets of dendritic cells (i.e., cDC₁, cDC₂, and plasmacytoid DC cells). Thus, the mice provided herein, which support a full range of major human immune cell populations, serve as better models of the human autoimmune response.

The humanized immunodeficient mouse models provided herein, in some aspects, are used as in vivo models to assess human autoimmunity, for example, systemic lupus erythematosus (e.g., via administration of pristane), inflammatory bowel disease (e.g., ulcerative colitis and Crohn's disease) (e.g., via administration of TNBS), multiple sclerosis (via administration of myelin), type 1 diabetes mellitus, psoriasis, and rheumatoid arthritis (via administration of collagen).

In some embodiments, a mouse model used to assess autoimmunity undergoes a myeloablative procedure, such as irradiation or chemical ablation.

In some embodiments, a mouse model used to assess autoimmunity is engrafted with huPBMCs (e.g., target drug naïve huPBMC). In some embodiments, an immunodeficient mouse is engrafted with 0.5×10⁶-50×10⁶ human PBMCs. For example, an immunodeficient mouse may be engrafted with 0.5×10⁶-50×10⁶, 1×10⁶-50×10⁶, 2×10⁶-50×10⁶, 5×10⁶-50×10⁶, 10×10⁶-50×10⁶, 15×10⁶-50×10⁶, 20×10⁶-50×10⁶, 0.5×10⁶-20×10⁶, 1×10⁶-20×10⁶, 2×10⁶-20×10⁶, 5×10⁶-20×10⁶, 10×10⁶-20×10⁶, 15×10⁶-20×10⁶, 0.5×10⁶-15×10⁶, 1×10⁶-15×10⁶, 2×10⁶-15×10⁶, 5×10⁶-15×10⁶, 10×10⁶-15×10⁶, 0.5×10⁶-10×10⁶, 1×10⁶-10×10⁶, 2×10⁶-10×10⁶, 5×10⁶-10×10⁶, 0.5×10⁶-5×10⁶, 1×10⁶-5×10⁶, 2×10⁶-10×10⁶, 0.5×10⁶-2×10⁶, 1×10⁶-2×10⁶, or 0.5×10⁶-1×10⁶ human PBMCs. In some embodiments, an immunodeficient mouse is engrafted with about 0.5×10⁶, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, about 9.5×10⁶, or about 10×10⁶ human PBMCs.

Following engraftment, an agent may be administered to facilitate the induction of a human autoimmune response. In some embodiments, a chemical agent such as 2,4,6-Trinitrobenzene sulfonic acid (TNBS) (e.g., in ethanol), dextran sulfate sodium (DSS), or oxazolone is administered (e.g., intrarectally) to mice, for example, to produce a model of human inflammatory bowel disease.

Trinitrobenzene sulfonic acid (TNBS) is a haptenating agent. It is a small molecule that causes an immune response when it binds to host proteins. TNBS administration results in a preclinical mouse model replicating clinical IBD. The immune response generated is Th1-mediated, characterized by infiltration of CD4+ T cells, neutrophils, and macrophages. Transversely-spreading inflammation develops, resulting in transmural colitis. TNBS-induced colitis models are ideal to study the immunologic aspects of IBD, for example, and, in some embodiments, to test the efficacy of potential new immunotherapies. TNBS is frequently used to induce a model of colitis in mice. This model is used to study inflammatory bowel diseases, such as Crohn's disease and ulcerative colitis. As a non-limiting example of how the model is prepared, TNBS is dissolved in a suitable solvent, usually ethanol. The concentration may vary, but it's typically around 2.5% to 5%. The ethanol helps to increase the permeability of the gut wall and to initiate an inflammatory response. The mice are fasted for several hours before the procedure. Under anesthesia, a catheter is inserted rectally and the TNBS solution is instilled into the colon. The volume administered can vary, but it's usually around 0.1 mL. The mice are allowed to recover and are typically kept in a head-down position for a few minutes to prevent leakage of the solution. Over the following days, the TNBS reacts with proteins in the colon, forming haptens that are recognized as foreign by the immune system. This leads to an immune response and inflammation in the colon. The mice are monitored for signs of colitis, which can include weight loss, changes in stool consistency, and presence of blood in the stool. After a suitable period (usually a few days to a week), the mice can be euthanized and the colon examined for signs of inflammation and damage.

DSS is a negatively charged sulfated polysaccharide that damages epithelial cells when administered to mice. Innate immune cells then release cytokines causing inflammation in the colon, characterized by ulcers and granulocyte infiltration. Common uses for the DSS-induced colitis model include studying how the innate immune system is involved in intestinal inflammation, and also for looking at factors that maintain or reestablish epithelium integrity during/after injury. DSS-induced colitis mouse models also respond to cyclosporine A, providing a relevant model, in some embodiments, to assess new agents that target the same immune mechanisms, e.g., new immunosuppressants.

Oxazolone is also a haptenating agent, but induces a different kind of inflammation to TNBS. This results in a model more similar to clinical ulcerative colitis, including similarities in immunopathogenesis. The immune response induced is Th2-mediated, resulting in diffuse colonic inflammation. In some embodiments, this model is used to study delayed-type hypersensitivity reactions in the skin, and in other embodiments, is used to assess agents targeting Th2-mediated mechanisms.

In other embodiments, pristane is administered to mice, for example, to produce a model of human lupus. Pristane is a naturally occurring hydrocarbon found in mineral oil. It has been used in scientific research to induce a model of lupus in mice, known as pristane-induced lupus (PIL). Pristane is believed to trigger an autoimmune response in susceptible strains of mice, leading to the development of lupus-like symptoms. In the pristane-induced lupus model, researchers inject pristane into mice, typically intraperitoneally (into the abdominal cavity). The injection of pristane stimulates the immune system and causes the production of autoantibodies, which are antibodies that mistakenly target the body's own cells and tissues. As a non-limiting example of how the model is prepared, pristane is obtained in a pure form and doesn't require much preparation. The mice receive a single intraperitoneal (IP) injection of pristane. The dosage can vary, but it's usually around 0.5 mL. Over the following weeks and months, the mice develop a lupus-like syndrome. The pristane stimulates the production of various autoantibodies, similar to those seen in human lupus. It also causes chronic inflammation in the peritoneum (the lining of the abdominal cavity), which mimics some of the inflammatory processes seen in lupus. The mice are monitored over time for signs of lupus, such as the production of autoantibodies and the development of immune complex deposits in the kidneys (glomerulonephritis). Other signs of lupus, like skin lesions, are typically not seen in this model.

In other embodiments, myelin is administered to mice, for example, to produce a model of human multiple sclerosis. The most common model of multiple sclerosis (MS) in mice is Experimental Autoimmune Encephalomyelitis (EAE). MS is a chronic autoimmune disease that targets the central nervous system, causing inflammation and damage to the myelin sheath that surrounds neurons. As a non-limiting example of how the model is prepared, rather than using whole myelin, specific peptides derived from myelin proteins are often used, such as myelin oligodendrocyte glycoprotein (MOG), proteolipid protein (PLP), or myelin basic protein (MBP). These peptides are dissolved in an appropriate buffer. The myelin peptide solution is emulsified with an adjuvant, often Complete Freund's Adjuvant (CFA), which contains killed bacteria (typically Mycobacterium tuberculosis). The purpose of the adjuvant is to boost the immune response. Sometimes, an additional immune stimulant, such as pertussis toxin, is administered. Mice are then immunized by injecting the emulsified peptide solution subcutaneously or intradermally. This injection causes the immune system to recognize the myelin peptides as foreign. Over the following days and weeks, the mice develop a condition similar to MS, with the immune system attacking the myelin in the central nervous system. This leads to inflammation and damage, resulting in clinical signs such as weakness and paralysis. The progression of EAE is then monitored over time, typically by observing the clinical signs and scoring the severity of the disease. At the end of the experiment, tissues can be analyzed to understand the immune response and assess the extent of the damage.

In other embodiments, Type II collagen is administered to mice, for example, to produce a model of human rheumatoid arthritis. Collagen-induced arthritis (CIA) is a commonly used animal model to study rheumatoid arthritis. This model has proven to be highly valuable in investigating the pathophysiological processes of arthritis and in evaluating potential therapeutic interventions. As a non-limiting example of how the model is prepared, Type II collagen, typically bovine or chicken, is dissolved in an acidic solution (often acetic acid) to create the collagen solution. The collagen solution is then emulsified with an adjuvant, often Complete Freund's Adjuvant (CFA), which contains killed bacteria (typically Mycobacterium tuberculosis). The purpose of the adjuvant is to boost the immune response. Mice are then immunized by injecting the emulsified collagen solution, usually into the base of the tail or into the flank. The injection causes the immune system to react against the collagen, which is similar to the mouse's own type II collagen. After about 21 days, a booster shot is often given to strengthen the immune response. This usually involves the same emulsified collagen solution, but sometimes without the adjuvant. After another couple of weeks, the mice typically start to develop signs of arthritis, such as swelling and redness in the joints, particularly in the hind paws. This is due to the immune system attacking the body's own collagen in the joints, which is seen as foreign due to the earlier exposure to the collagen solution. The progression of arthritis is then monitored over time, and the mice can be analyzed to understand the immune response and disease progression.

In some embodiments, the agent is administered to the mouse about 4 to 10 days following administration of the huPBMCs. For example, the agent may be administered 4, 5, 6, 7, 8, 9, or 10 days following administration of the huPBMCs. In some embodiments, the agent is administered to the mouse at least 4 days, at least 5 days, at least 6 days, at least 7 days, at least 8 days, at least 9 days, or at least 10 days following administration of the huPBMCs.

In some embodiments, a therapeutic agent is administered (e.g., prior to administration of the chemical agent or beginning about 6-24 hours post administration of the chemical agent).

Autoimmunity in the mouse models provided herein may be assessed over a period of time of about 3 to about 21 days, for example, daily or every other. For example, mice may be monitored daily, recording body condition score, fecal score, and body weight. Following the monitoring period, mice may be euthanized, colons weighed, and colon length measured prior to fixing for histology, for example.

### Delayed-Type Hypersensitivity (DTH)

Some aspects provide method of using the mouse models described herein to assess delayed-type hypersensitivity (DTH) reactions, also known as type IV hypersensitivity reactions. DTH reactions are antigen-specific cell-mediated immune responses that can invoke harmful aspects of immune function (e.g., allergic dermatitis and autoimmunity). DTH reactions are mediated by soluble or cell-associated antigens primarily involving CD4⁺ or CD8⁺ T cell activation. These reactions are characterized by the release of mediators from activated T cells. The T cells then activate local endothelial cells and recruit macrophages, which results in local inflammation and swelling. There are three major categories of DTH reactions, classified by delivery of the antigen: injected into the skin, absorbed into the skin, or absorbed through the gut.

The humanized immunodeficient mouse models provided herein, in some aspects, are used as in vivo models to assess a DTH reaction.

In some embodiments, a mouse model used to assess a DTH reaction undergoes a myeloablative procedure, such as irradiation or chemical ablation.

In some embodiments, a mouse model used to assess a DTH reaction is engrafted with huPBMCs (e.g., target drug naïve huPBMC). In some embodiments, an immunodeficient mouse is engrafted with 0.5×10⁶-50×10⁶ human PBMCs. For example, an immunodeficient mouse may be engrafted with 0.5×10⁶-50×10⁶, 1×10⁶-50×10⁶, 2×10⁶-50×10⁶, 5×10⁶-50×10⁶, 10×10⁶-50×10⁶, 15×10⁶-50×10⁶, 20×10⁶-50×10⁶, 0.5×10⁶-20×10⁶, 1×10⁶-20×10⁶, 2×10⁶-20×10⁶, 5×10⁶-20×10⁶, 10×10⁶-20×10⁶, 15×10⁶-20×10⁶, 0.5×10⁶-15×10⁶, 1×10⁶-15×10⁶, 2×10⁶-15×10⁶, 5×10⁶-15×10⁶, 10×10⁶-15×10⁶, 0.5×10⁶-10×10⁶, 1×10⁶-10×10⁶, 2×10⁶-10×10⁶, 5×10⁶-10×10⁶, 0.5×10⁶-5×10⁶, 1×10⁶-5×10⁶, 2×10⁶-10×10⁶, 0.5×10⁶-2×10⁶, 1×10⁶-2×10⁶, or 0.5×10⁶-1×10⁶ human PBMCs. In some embodiments, an immunodeficient mouse is engrafted with about 0.5×10⁶, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, about 9.5×10⁶, or about 10×10⁶ human PBMCs.

Following engraftment, an agent may be administered to facilitate sensitization. In some embodiments, a chemical agent such as 2,4-Dinitro-1-fluorobenzene (DNFB) (e.g., 0.5%), oxazolone, or keyhole limpet hemocyanin (KLH) is administered (e.g., intraperitoneally) to sensitize the mouse. In some embodiments, the agent is administered to the mouse about 4 to 10 days following administration of the huPBMCs. For example, the agent may be administered 4, 5, 6, 7, 8, 9, or 10 days following administration of the huPBMCs. In some embodiments, the agent is administered to the mouse at least 4 days, at least 5 days, at least 6 days, at least 7 days, at least 8 days, at least 9 days, or at least 10 days following administration of the huPBMCs. A mouse, in some embodiments, is sensitized one or more times, using the same chemical agent, for example, DNFB. In some embodiments, a second sensitization occurs about 2 to about 5 days (e.g., about 2, 3, 4, or 5 days) following a first sensitization (e.g., intraperitoneal administration of the chemical agent, such as 0.5% DNFB).

Following sensitization, in some embodiments, a therapeutic agent is administered (e.g., after sensitization and before challenge or beginning about 12-24 hours post challenge). Efficacy endpoints include but are not limited to PK/PD blood collections, cytokine/chemokine analysis, clinical chemistry, histopathological evaluation, immunohistochemistry, and flow cytometry of lymph nodes or spleen. In some embodiments, efficacy is determined by differences in ear thickness. In some embodiments, efficacy is determined by differences in serum cytokine profile. In some embodiments, efficacy is determined by histology of an affected tissue.

Sensitized mice may then be challenged by administration of the chemical agent, for example, at a lower concentration and/or dose (e.g., 0.2% DNFB). In some embodiments, a mouse is challenged by administering the chemical agent to the dorsal and/or ventral side of one ear, while the other ear serves as a vehicle control (e.g., carrier without chemical agent).

A DTH reaction in the mouse models provided herein may be assessed over a period of time, for example, daily or every other day for a period of about 3 days to about 2 weeks or longer.

Assessment of DTH reaction includes, in some embodiments, assessing inflammation, for example, cytokine/chemokine release by T cells. In some embodiments, ear thickness of a mouse is assessed. Other endpoints include PK/PD blood collections, clinical chemistry, histopathological evaluation, immunohistochemistry, and/or flow cytometry in lymph nodes or spleen.

### EXAMPLES

### Example 1. Irradiated NSG-Flt3^{null} (Hu-FLT3L) huPBMC-Engrafted Mice Display Higher Levels of CD14+ Monocytes in the Blood and Lungs Compared to NSG-SGM3 (Hu-IL-15) huPBMC-Engrafted Mice

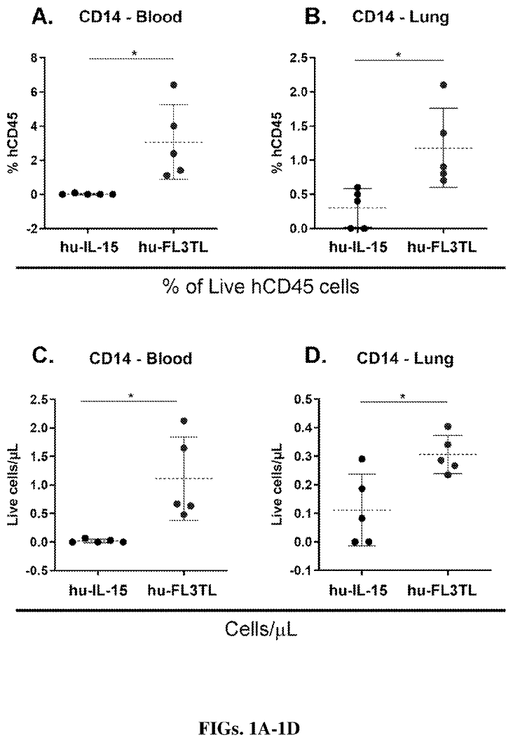

Irradiated NSG-Flt3^{null} (hu-FLT3L) (The Jackson Laboratory Strain #: 033367, common name NSG Flt3KO hFLT3LG Tg) and NSG-SGM3 (hu-IL-15) (The Jackson Laboratory Strain #: 033216, common name NSG-SGM3-IL15) mice were engrafted with 10 million huPBMC each from donor 9348 on study day 0. Five days later mice were euthanized, blood and lungs were collected and processed into single cell suspensions. Cells were stained for flow cytometry and number of CD14+ cells reported. Frequency of CD14 was measured as a percentage of human CD45 in blood (FIG. 1A) and lungs (FIG. 1B). Live number of CD14+ cells was measured per μL blood (FIG. 1C) and per μL of single cell suspension from lung dissociation (FIG. 1D). Data is presented as dot plot graphs; bars represent mean and standard deviation. * denotes p-value less than 0.05 (n=5). CD14 is highly expressed on monocytes and macrophages. These data demonstrate that the humanized FLT3L mouse model is capable of supporting higher numbers of human monocyte/macrophage cells than NSG-SGM3 (hu-IL-15). In addition, the data also demonstrate CD14+ cells are not only present in the blood but also migrate to tissues.

### Example 2. Irradiated NSG-Flt3^{null} (Hu-FLT3L) huPBMC-Engrafted Mice Support Expansion of Major Immune Populations Including B Cells and Myeloid Subsets

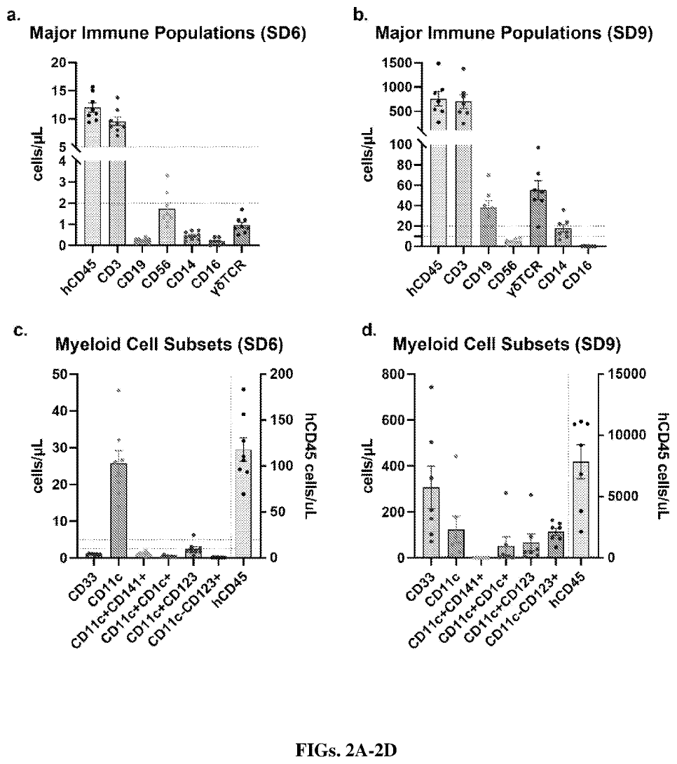

Irradiated NSG-Flt3^{null} (hu-FLT3L) mice were engrafted with 30 million huPBMC from donor 7904 (n=16). Blood was collected via cardiac puncture 6 or 9 days post engraftment, and flow cytometry analysis was performed. NSG-Flt3^{null} (hu-FLT3L) huPBMC-engrafted mice support expansion of T cells (CD3+), including 76 T cells, B cells (CD19+), and monocytes (CD14+) (FIG. 2A, 2B). Dendritic cell (DC) populations, including conventional (cDC) DC1 (CD11c+CD141+), cDC2 (CD11c+CD1c+) and plasmacytoid DC (CD11c−CD123+), were also found in the blood 6 days post engraftment (FIG. 2C) with expansion 3 days later (FIG. 2D) All immune cell populations were gated based on human CD45 expression. Dendritic cell populations were first gated as lineage negative (CD3−CD19−CD56−) before analyzing CD11c expression. 100 μL of blood from each animal was stained for major immune cell populations (FIG. 2A, 2B) and the remaining 300+μL of blood was stained for myeloid cell populations (FIG. 2C, 2D). Cells were analyzed by flow cytometry with counting beads to determine cells per μL. These data show that not only are the NSG-Flt3^{null} (hu-FLT3L) mice capable of supporting PBMC myeloid cell populations, but these cells are also able to proliferate in the NSG-Flt3^{null} (hu-FLT3L) mouse, which has not been show in the other huPBMC-humanized mice.

### Example 3. Irradiated NSG-Flt3^{null} (Hu-FLT3L) Mice are Engrafted with Lower Number of huPBMC and Display High Levels of B Cells in the Bone Marrow

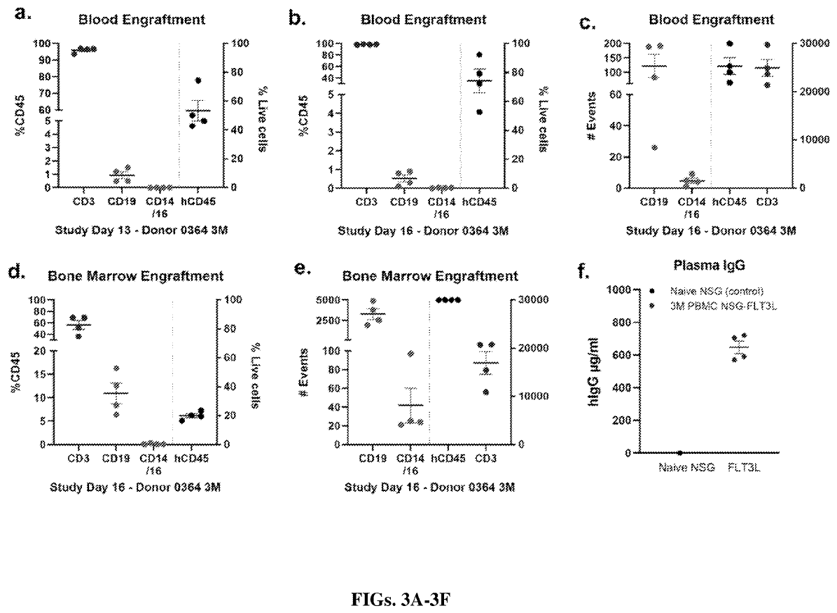

Irradiated NSG-Flt3^{null} (hu-FLT3L) mice were engrafted with 3 million huPBMC from donor 0364 (n=4). Terminal blood was collected via cardiac puncture 13 days (FIG. 3A) and 16 days (FIGS. 3B, 3C) post-engraftment, and bone marrow was flushed from both femurs on study day 16 (FIGS. 3D, 3E). Cells were stained for flow cytometry analysis. NSG-Flt3^{null} (hu-FLT3L) mice showed high percentage (42-74% of live cells in circulation) of hCD45 engraftment by 13 days post engraftment (FIG. 3A) and showed a substantial population of B cells in the bone marrow (6-16% of hCD45 cells, 1925-4885 events) identified by CD19 expression (FIGS. 3D, 3E, circles). Human immunoglobulin G was also detected in NSG-Flt3^{null} (hu-FLT3L) mice engrafted with 3 million huPBMC 16 days post engraftment (plasma concentration averaged 647 μg/mL; FIG. 3F), suggesting the presence of functional plasma cells in the bone marrow. While CD14+ monocyte numbers are comparatively lower (FIGS. 3C, 3E, circles) they are not usually found in NSG® and NSG-MHCI/MHCII knockout mice 6-8 days post engraftment with huPBMCs.

### Example 4. Irradiated NSG-Flt3^{null} (Hu-FLT3L) huPBMC-Engrafted Mice Display High Levels of Monocyte Relevant Cytokines

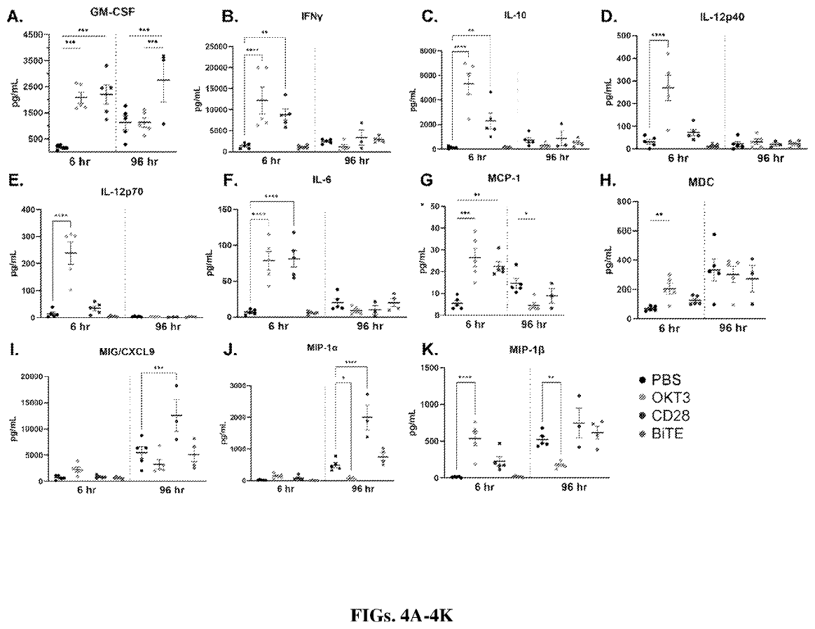

Irradiated NSG-Flt3^{null} (hu-FLT3L) mice were engrafted with 15 million huPBMC from donor 7904 on study day 0. On study day 6 mice were treated with PBS, OKT3, anti-CD28 antibody, or CD19×CD3 bispecific T cell engager to activate the human immune cells. Blood was collected and processed to serum 6 hours post-dosing and again at 96 hours post-dosing. Serum was frozen immediately after collection and stored at −80° C. until cytokine analysis. Cytokines were quantified by a Luminex multiplex assay measuring 48 human cytokines. Monocyte relevant cytokines are depicted as dot plots graphs; bars represent mean and standard deviation (FIGS. 4A-4K). Statistical significance was determined by One-way ANOVAs comparing each time point separately. * denotes p-value less than 0.05, **p-value less than 0.01, ***p-value less than 0.001, ****p-value less than 0.0001. These data demonstrate that NSG-Flt3null (hu-FLT3L) mice are able to produce a robust cytokine response to T cell agonists. In particular, cytokines, chemokines, and growth factors that are primarily produced by myeloid cells are shown to be elevated. Myeloid cells play an important role in bridging the gap between innate and adaptive immunity.

### Example 5. Irradiated NSG-Flt3^{null} (Hu-FLT3L) Mice Humanize Faster and have Larger Expansion of B Cells Compared to Non-Irradiated NSG-Flt3^{null} (Hu-FLT3L) Mice

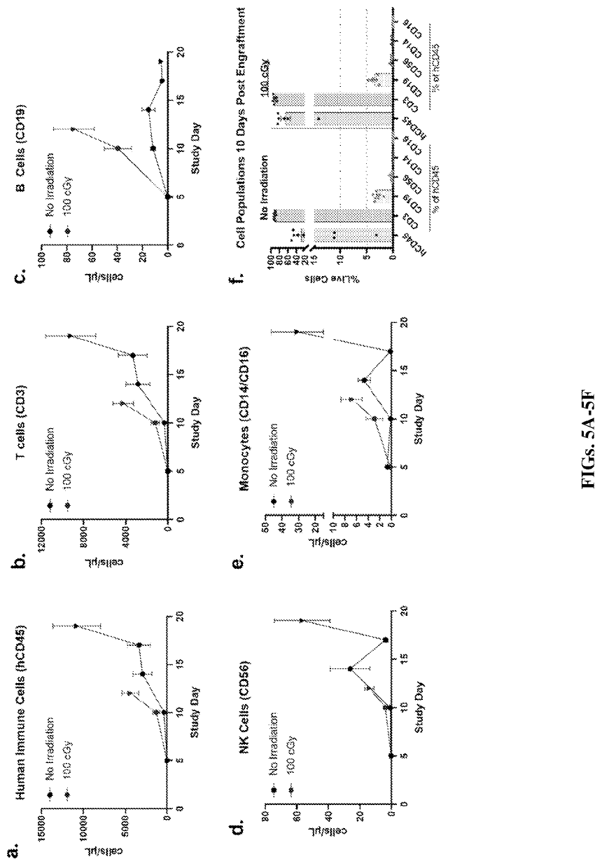

NSG-Flt3null (hu-FLT3L) mice were engrafted with 10 million huPBMC with or without 100 cGY irradiation (n=16) and placed on alternating bleed groups to accommodate regular retro-orbital (RO) bleeds. Mice were retro-orbitally bled 5, 10, 14, or 17 days post engraftment (depicted as circles) and terminally bled on either study day 12 (irradiated mice) or study day 19 (non-irradiated) (depicted with triangle). 70 μL of blood was stained per mouse and cells were analyzed by flow cytometry with counting beads to determine cells per μL (FIGS. 5A-5F). Major immune cell populations occur at a similar frequency of human CD45 in irradiated and non-irradiated NSG-Flt3^{null} (hu-FLT3L) huPBMC-engrafted mice; however, total numbers of human CD45 cells (FIG. 5A) and frequency of total live cells (FIG. 5F, gray bar) are higher in irradiated mice. B cells in particular are shown to increase at a faster pace than nonirradiated mice, and while the non-irradiated mice are able to survive longer likely due to delayed GvHD, B cells populations begin to shrink in the non-irradiated mice after study day 14 (FIG. 5C).

### Example 6. NSG-Flt3^{null} (Hu-FLT3L) Mice Exhibit Long-Term huPBMC Engraftment with or without Irradiation

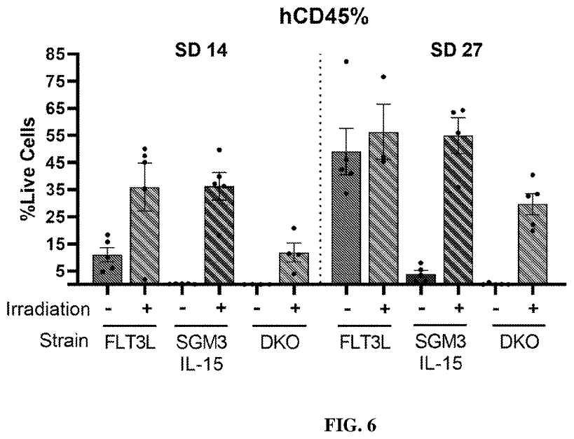

NSG-Flt3^{null} (hu-FLT3L), NSG-SGM3 (hu-IL-15), and NSG-MHCI/MHCII knockout (DKO) mice were engrafted with 4 million huPBMC from donor 0595 on study day 0, with or without 100 cGY irradiation (n=5 per group). Mice were retro-orbitally bled 14 days post engraftment and terminally bled on study day 27. Cells were analyzed by flow cytometry with counting beads to determine cells per μL. Total numbers of human CD45 and frequency of total live cells were measured as a readout of engraftment. As expected, all three strains showed substantial engraftment when irradiated with 100 cGY. However, only NSG-Flt3^{null} (hu-FLT3L) mice also showed successful engraftment and expansion of huCD45+ cells without irradiation (FIG. 6).

### Example 7. B Cell Expansions in NSG-Flt3^{null} (Hu-FLT3L) PBMC-Engrafted Mice Show Class Switching Capabilities with or without Irradiation

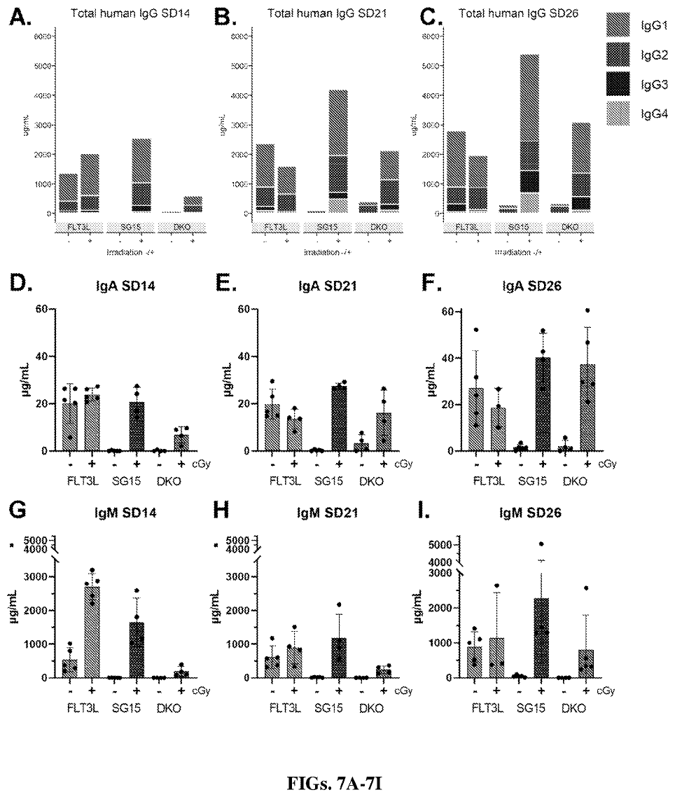

NSG-Flt3^{null} (hu-FLT3L), NSG-SGM3 (hu-IL-15), and NSG-MHCI/MHCII knockout (DKO) mice were engrafted with 4 million PBMC from donor 0595 on study day 0. Total levels of human immunoglobulin G (IgG), immunoglobulin A (IgA) and immunoglobulin M (IgM) were measured on days 14, 21, and 26 post-engraftment as a readout of B cell maturation and function. Naïve B cells produce IgM, and upon exposure specific signaling molecules undergo antibody class switching to produce IgG and IgA. While all three strains showed production of IgM, IgG, and IgA when irradiated, only NSG-Flt3^{null} (hu-FLT3L) mice showed different Ig isotypes without irradiation (FIGS. 7A-7I).

### Example 8. HuPBMC CD3 Depletion Reveals Preservation of Functionally Active B Cells in NSG-FLT3L Mice

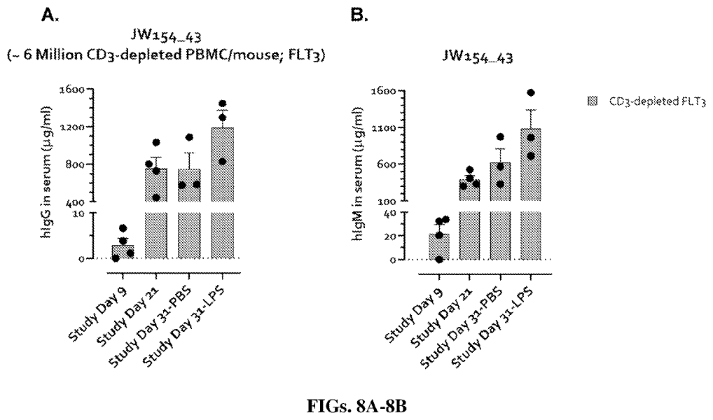

Irradiated NSG-Flt3^{null} (hu-FLT3L) mice were engrafted with 6 million huPBMC without expression of CD3, an important co-receptor necessary for T cell activation. Blood was collected and processed to serum on days 9 and 21 post-engraftment. On study day 27, mice were separated into two groups for dosing: Group 1 received a dose of PBS, and Group 2 received a dose of lipopolysaccharide (LPS) to stimulate IgG and IgM production. 4 days later (study day 31), blood was collected from both groups and processed to serum. Serum levels of human IgG (FIG. 8A) and IgM (FIG. 8B) were quantified by ELISA. Even in mice engrafted with CD3-depleted huPBMCs, LPS stimulated production of IgG and IgM, indicating preservation of functionally active B cells in NSG-Flt3^{null} (hu-FLT3L) mice.

### Example 9. Immune Cell Populations in NSG-Flt3^{null} (Hu-FLT3L) and NSG-SGM3 (Hu-IL-15) Mice Engrafted with huPBMC

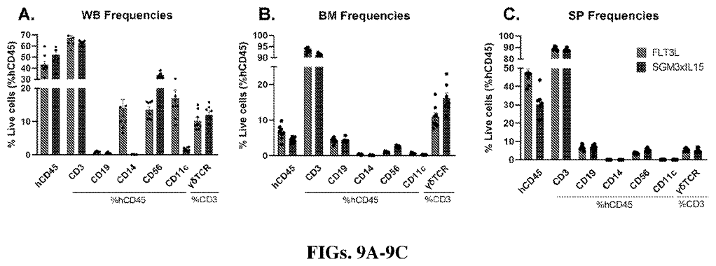

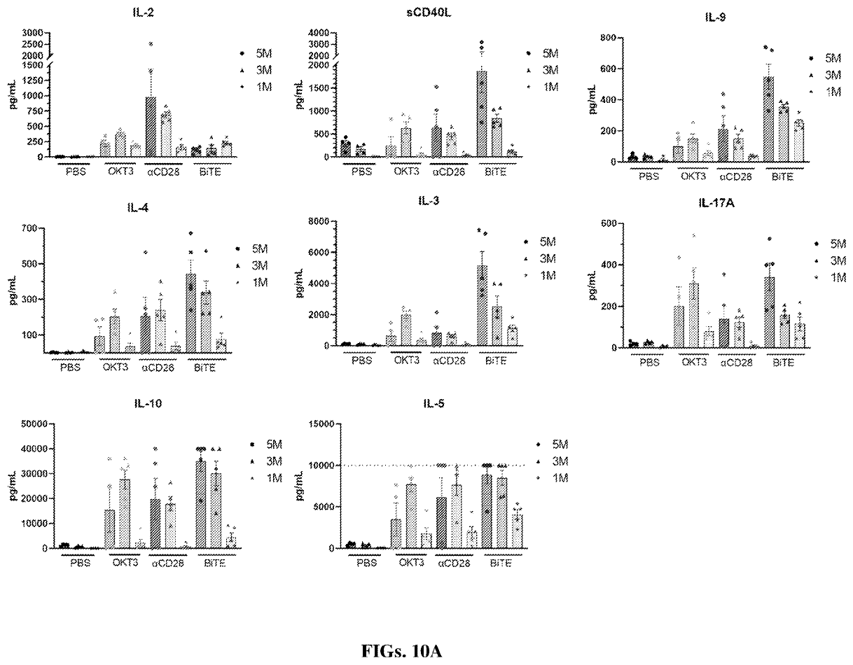

NSG-Flt3^{null} (hu-FLT3L) and NSG-SGM3 (hu-IL-15) mice were irradiated with 100 cGy and engrafted with 30 million huPBMC. On study day 5, mice were euthanized; blood and spleen were collected, bone marrow was flushed from both femurs, and all samples processed into single cell suspensions. Cells were stained for flow cytometry and number of hCD45+ and hCD3+ cells measured. Frequency of CD45 and CD3 was measured as a percentage of total live cells in the blood (FIG. 9A), bone marrow (FIG. 9B), and spleen (FIG. 9C). While both mouse strains had similar levels of γδ T cells, hu-FLT3L mice had a higher frequency of hCD45+ cells in the bone marrow and spleen.

### Example 10. NSG-Flt3^{null} (Hu-FLT3L) Mice Engrafted with 1, 3, or 5 Million huPBMC Produce Monocyte Relevant Cytokine Levels in a huPBMC Dose-Dependent Manner

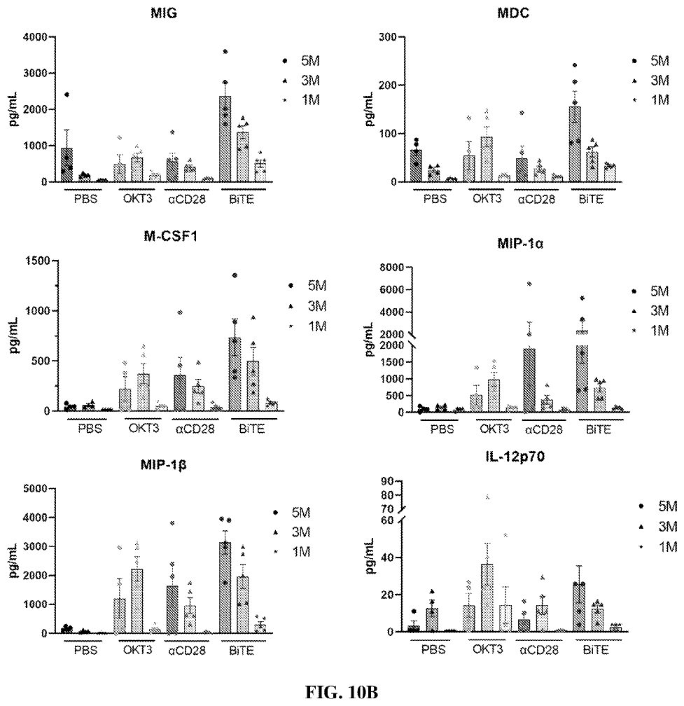

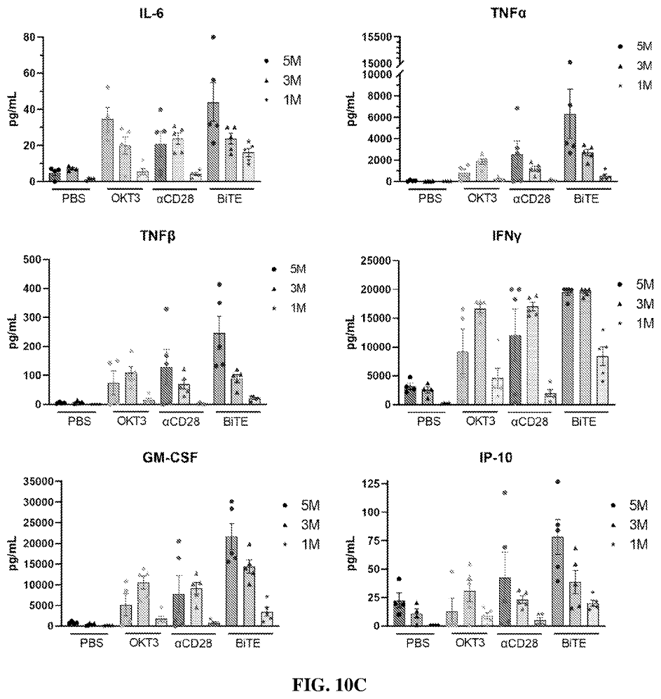

NSG-Flt3^{null} (hu-FLT3L) were irradiated with 100 cGy and engrafted with 1, 3, or 5 million huPBMC. Mice were grouped based on bodyweight and dosed with PBS, OKT3, anti-CD28 antibody, or CD19×CD3 bispecific T cell engager on study day 9 (n=4-5 per group). Blood was collected and processed to serum 6 hours post-dosing. Serum was frozen immediately after collection and stored at −80° C. until cytokine analysis. Cytokines were quantified and grouped based on the immune cell type with which they are associated: T cells (FIG. 10A), monocytes (FIG. 10B) or innate immune cells (FIG. 10C). All cytokines showed increased expression in huPBMC-engrafted mice, with the majority of cytokines showing a dose-dependent response depending on the amount of huPBMC engrafted.

### Example 11. NSG-Flt3^{null} (Hu-FLT3L) Mice Engrafted with 5, 3, or 1 Million huPBMC and Produce IgG and IgM in a PBMC Dose-Dependent Fashion

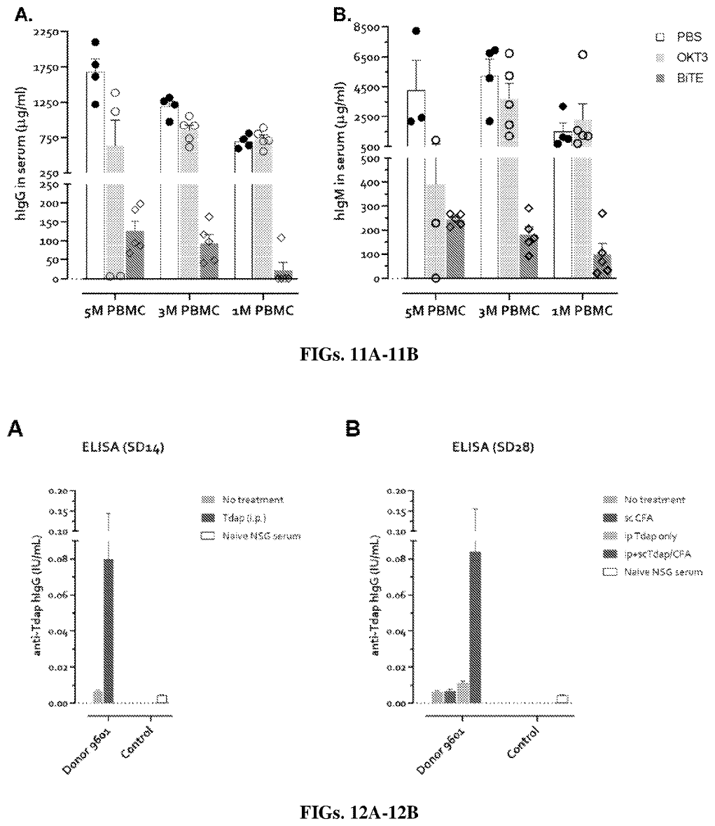

Irradiated NSG-Flt3^{null} (hu-FLT3L) mice were engrafted with 5, 3, or 1 million huPBMC from donor 3875. Mice were grouped based on bodyweight and dosed with PBS, OKT3, or CD19×CD3 bispecific T cell engager on study day 9 (n=3-5 per group). Terminal blood was collected via cardiac puncture on study day 12 and processed to serum. Serum levels of human immunoglobulin (Ig) G (FIG. 11A) and IgM (FIG. 11B) were quantified by ELISA. Mice who received no drug (PBS) in general displayed higher levels of IgG compared to the huPBMC dose equivalent OKT3 and bispecific T cell engager groups, and when looking only at the PBS group, IgG and IgM levels decrease with decreasing levels of huPBMC. When animals were treated with the bispecific T cell engager, a bispecific antibody designed to target the elimination of CD19-expressing cells (B cells), there was a dramatic drop in both IgG and IgM levels, demonstrating that circulating immunoglobulin levels are actively being produced by B cells and are not from the early timepoints of huPBMC engraftment.

TABLE 1
Human cytokines measured from NSG-Flt3^{null} (hu-FLT3L) huPBMC-engrafted
mouse serum 6 and 96 hours post-dosing with OKT3 and anti-CD28 antibody
Human Serum Cytokines

| | 6 hr post-dosing | | | 96 hr post-dosing | | |
|---|---|---|---|---|---|---|
| | PBS | OKT3 | CD28 | PBS | OKT3 | CD28 |
| EGF | 2.871 ± 1.557 | 3.092 ± 0.6007 | 2.99 ± 1.278 | 4.383 ± 2.646 | ND | ND |
| Eotaxin | 22.61 ± 6.057 | 27.12 ± 5.148 | 24.3 ± 4.555 | 13.7 ± 7.683 | 11.03 ± 9.852 | 17.57 ± 8.622 |
| FGF-2 | 49.15 ± 64.58 | 45.12 ± 31.93 | 36.85 ± 21.33 | 640.5 ± 1009 | 1788 ± 3052 | 2482 ± 3865 |
| FLT3 | 6887 ± 2348 | 4960 ± 1667 | 5614 ± 934.8 | 2940 ± 2395 | 2099 ± 1265 | 2600 ± 677.2 |
| Fractalkine | ND | ND | ND | ND | ND | ND |
| G-CSF | 7.433 ± 5.887 | 11.72 ± 15.47 | 11.31 ± 13.02 | ND | ND | 6.355 ± 2.563 |
| GM-CSF | 175.5 ± 81.97 | 2086 ± 483.7* | 2203 ± 821.1^{†} | 1119 ± 591.5 | 1131 ± 417.2 | 2758 ± 1463^{†‡} |
| GROα | 2.509 ± 3.28 | 2.463 ± 1.019 | 1.596 ± 1.166 | 1.09 ± 0.959 | 3.959 ± 4.063 | 1.231 ± 0.04258 |
| IFNα | 37.31 ± 46.63 | 23.04 ± 4.194 | 9.935 ± 4.013 | 9.271 ± 5.533 | 6.429 ± 3.512 | 6.201 ± 3.116 |
| IFNγ | 1421 ± 652.1 | 12196 ± 7146* | 8827 ± 2938 | 2493 ± 550.7 | 1182 ± 1079 | 3387 ± 3128 |
| IL-10 | 139.6 ± 94.19 | 5332 ± 1894* | 2306 ± 1418^{‡} | 740 ± 455.4 | 293.1 ± 196.9 | 882.9 ± 1037 |
| IL-12p40 | 31.59 ± 23.79 | 269.1 ± 125.4* | 72.05 ± 32.23^{‡} | 21.55 ± 23.93 | 31.61 ± 27.52 | 19.92 ± 13.46 |
| IL-12p70 | 14.42 ± 14.28 | 239 ± 93.2* | 34.25 ± 20.45^{‡} | 4.32 ± 1.584 | ND | 2.624 ± 0.9973 |
| IL-13 | 235.5 ± 122.3 | 1431 ± 363.4* | 1605 ± 562.6^{†} | 322.5 ± 344.2 | 159.2 ± 179.9 | 1419 ± 516^{†‡} |
| IL-15 | 1.02 ± 0.656 | 2.02 ± 1.137 | 1.478 ± 0.3668 | 1.657 ± 1.642 | 1.789 ± 1.48 | 0.9515 ± 0.5988 |
| IL-17A | 1.229 ± 0.113 | 56.06 ± 24.55* | 15.82 ± 7.649^{‡} | 6.617 ± 8.122 | 3.488 ± 3.883 | 8.147 ± 12.11 |
| IL-17E | ND | ND | ND | ND | ND | ND |
| IL-17F | 4.986 ± 2.697 | 10.81 ± 4.41 | 9.354 ± 4.246 | 12.64 ± 13.32 | 12.99 ± 13.44 | 20.51 ± 25.1 |
| IL-18 | ND | 0.688 ± 0.642 | 0.776 ± 0.293 | 1.40 ± 1.52 | 1.40 ± 1.45 | 2.29 ± 1.85 |
| IL-1α | 1.98 ± 0.788 | 3.743 ± 1.006* | 3.926 ± 1.089^{†} | 1.632 ± 1.178 | 1.261 ± 1.422 | 12.88 ± 15.93 |
| IL-1β | 0.917 ± 0.933 | 1.166 ± 0.7977 | 0.868 ± 0.686 | 3.663 ± 3.162 | 4.51 ± 3.864 | 1.728 ± 0.2224 |
| IL-RA | ND | ND | ND | 5.191 ± 3.013 | 1.602 ± 1.123 | 3.861 ± 2.452 |
| IL-2 | 4.481 ± 1.814 | 573.1 ± 220.2* | 973.8 ± 427.7^{†} | 0.815 ± 0.388 | 0.5016 ± 0.232 | 1.552 ± 1.58 |
| IL-22 | 10.76 ± 5.248 | 13.03 ± 2.379 | 10.21 ± 6.594 | 17.08 ± 9.562 | ND | 9.36 ± 5.958 |
| IL-27 | 15.48 ± 0.709 | 95.05 ± 16.72* | 67.77 ± 21.7^{†‡} | 381.5 ± 184.4 | 140.3 ± 77.41* | 141.6 ± 94.36 |
| IL-3 | 44.44 ± 19.36 | 863.7 ± 374.3* | 327.5 ± 164^{‡} | 243.5 ± 151.5 | 94.25 ± 65.81 | 1775 ± 1650^{†‡} |
| IL-4 | 1.419 ± 1.483 | 33.74 ± 36.44 | 44.28 ± 26.69 | 1.196 ± 0.772 | ND | 1.042 ± 0.6957 |
| IL-5 | 313.7 ± 122.7 | 2000 ± 668* | 2511 ± 496.8^{†} | 122.1 ± 73.6 | 75.86 ± 70.44 | 7685 ± 5452^{†‡} |
| IL-6 | 7.076 ± 3.552 | 78.36 ± 28.76* | 81.23 ± 25.71^{†} | 20.36 ± 11.42 | 8.916 ± 5.311 | 10.17 ± 10.49 |
| IL-7 | 0.906 ± 0.195 | 0.672 ± 0.0736 | 0.424 ± 0.156^{†} | 0.762 ± 0.332 | 0.554 ± 0.194 | 0.589 ± 0.0717 |
| IL-8 | 0.679 ± 0.249 | 7.742 ± 4.02* | 3.79 ± 1.605 | 32.19 ± 14.66 | 10.92 ± 4.382 | 43.02 ± 25.11^{‡} |
| IL-9 | 40.24 ± 19.11 | 72.87 ± 53.37 | 93.4 ± 45.06 | 16.25 ± 2.814 | ND | 344.1 ± 420.7 |
| IP-10 | 7.905 ± 3.322 | 21.08 ± 6.894* | 18.91 ± 4.689^{†} | 168.9 ± 89.49 | 72.16 ± 15.41 | 129.7 ± 94.57 |
| M-CSF | 8.967 ± 3.397 | 72.78 ± 35.49 | 111 ± 76.02^{†} | 632.9 ± 388.8 | 213.7 ± 98.81 | 79.03 ± 110.6^{†} |
| MCP-1 | 5.576 ± 2.826 | 26.58 ± 9.716* | 22.44 ± 4.88^{†} | 14.68 ± 4.97 | 4.6 ± 2.908* | 8.916 ± 5.723 |
| MCP-3 | 6.11 ± 5.001 | 22.02 ± 12.27* | 20.05 ± 7.137 | 13.89 ± 13.17 | ND | 12.17 ± 3.696 |
| MDC | 69.03 ± 17.08 | 204.8 ± 83.12* | 125.5 ± 30.57 | 333.7 ± 170.4 | 302.2 ± 124.8 | 273.1 ± 156.6 |
| MIG | 708 ± 401.3 | 2251 ± 1069* | 892.7 ± 256.7^{‡} | 5492 ± 2551 | 3267 ± 2009 | 12572 ± 5237^{†‡} |
| MIP-1α | 28.15 ± 10.57 | 151 ± 74.36* | 92.24 ± 65.22 | 493.7 ± 176.2 | 77.14 ± 38.68 | 1998 ± 685.7^{†‡} |
| MIP-1β | 11.82 ± 8.16 | 536 ± 223.9* | 225 ± 140.5^{‡} | 522 ± 101.1 | 175.8 ± 41.04* | 748.5 ± 352.5^{‡} |
| PDGF-AA | ND | ND | ND | 13.09 ± 0.653 | ND | ND |
| PDGF-AB | ND | ND | ND | ND | ND | ND |
| RANTES | 1.345 ± 1.05 | 2.418 ± 1.791 | 1.55 ± 1.272 | 234.4 ± 81.62 | 344.8 ± 170.3 | 273 ± 102.9 |
| sCD40L | 32.42 ± 17.86 | 130.2 ± 60.56* | 110.6 ± 40.74^{†} | 363.3 ± 109.3 | 324.8 ± 108.6 | 2161 ± 934.4^{†‡} |
| TGFα | ND | 1.476 ± 0.439 | 1.048 ± 0.518 | 1.871 ± 1.321 | 2.277 ± 2.229 | 2.264 ± 1.705 |
| TNFα | 33.63 ± 21.34 | 756.5 ± 279.8* | 712.8 ± 419.6^{†} | 134 ± 66.24 | 87.37 ± 39.23 | 649.3 ± 299.7^{†‡} |
| TNFβ | 16.04 ± 7.813 | 346.9 ± 117.3* | 356.3 ± 122.2^{†} | 23.34 ± 41.86 | 2.814 ± 1.964 | 13.26 ± 10.16 |
| VEGF | 33.67 ± 21.14 | 39.12 ± 21.3 | 49.13 ± 26.39 | 3.108 ± 1.302 | 4.02 ± 1.999 | 4.037 ± 2.558 |

### Example 12. A Modified Tdap Vaccination Protocol of NSG-Flt3^{null} (Hu-FLT3L) Mice Induces Tetanus Toxoid Antibody Production and Increases the Number of Engrafted Human Cells

8-week-old NSG-Flt3^{null} (hu-FLT3L) mice were engrafted intraperitoneally with either 4×10⁶ huPBMC (Donor 9601) or huPBMC mixed with Tdap vaccine (50 μL/mL). Plasma isolated 14 days post-engraftment (SD14) was analyzed for the presence of human anti-tetanus toxoid IgG by ELISA (FIG. 12A; n=15 mice/group). On SD14, different cohorts of mice were injected subcutaneously (s.c.) with either CFA or Tdap/CFA. Control mice were injected s.c. with PBS only. Two weeks after the booster (SD28), mice were euthanized and collected serum was analyzed for anti-tetanus toxoid IgG as in FIG. 12A (FIG. 12B; n=5 mice/group; Bars are plotted as SEM).

Tetanus toxoid immune response was initiated when huPBMC were pre-mixed with Tdap vaccine at the time of engraftment. Though the booster shot does not increase the levels of tetanus toxoid antibodies, it prevents the decline of antigen-specific IgG which occurs between 2 and 4 weeks post-initial immunization.

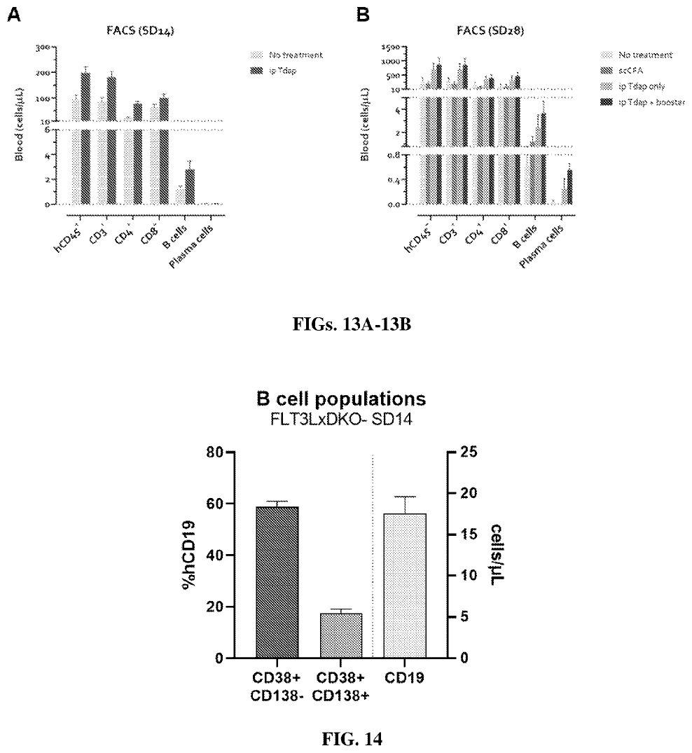

Mouse blood was collected on SD14 and analyzed by FACS; n=15 mice/group (FIG. 13A). On SD14, different cohorts of mice were injected subcutaneously (s.c.) with either CFA or Tdap/CFA (FIG. 13B). Control mice were injected s.c. with PBS only. Two weeks after the booster (SD28), blood was analyzed again as in A); n=5 mice/group; Bars are plotted as SEM.

Tdap vaccination results in detectible increase in human leukocyte populations, most notably for B cells. Strikingly, on SD28, plasma cells are clearly detected in the blood of Tdap vaccinated animals but not in control mice.

### Example 13. TDaP Vaccination and Challenge Studies

NSG-Flt3^{null} (hu-FLT3L) mice in which MHC Class I and MHC Class II genes were knocked out were (herein “NSG-Flt3^{null} (hu-FLT3L) DKO”, also referred to as “FLT3LxDKO” mice) were engrafted with 10 million human PBMCs and were bled 2 weeks later. Whole blood was stained and analyzed by flow cytometry. The right axis of FIG. 14 shows the absolute cell counts of hCD19 in cells/μL and the left axis of the graph depicts the percentage of hCD19 cells that express the activation marker CD38, which is associated with activated B cells and plasmablasts, and the plasma cell marker CD138.

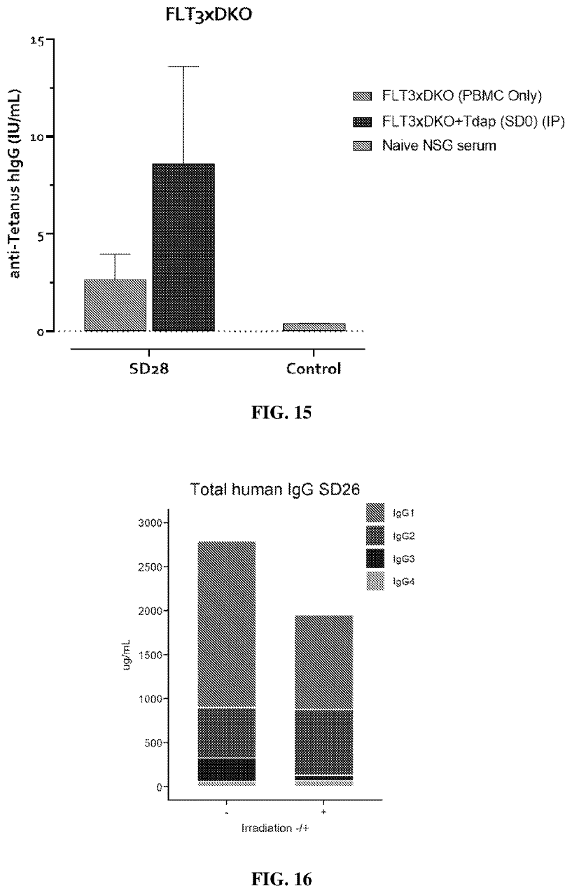

NSG-Flt3^{null} (hu-FLT3L) DKO mice were then engrafted with 10 million human PBMCs (control) or engrafted with a mixture of 10 million human PBMC and 50 μL/mL TDaP vaccine. Serum from the mice was collected and analyzed for anti-tetanus toxoid hIgG titers at 4 weeks post challenge with TDaP. Vaccinated mice challenged with TDaP produced higher levels of human anti-tetanus antibodies compared to mice who did not receive TDaP vaccine (FIG. 15). Naïve NSG serum was used as a negative control and shows little to no signal.

### Example 14. Irradiation Studies

NSG-Flt3^{null} (hu-FLT3L) mice were engrafted with 4 million human PBMC with or without 100 cGy of irradiation and bled once a week. Serum was analyzed for the four IgG subclasses: IgG1, IgG2, IgG3, IgG4. Human IgG is shown as a stacked bar graph with each color bar depicting a different Ig isotype. The ratio of the four human IgG subclasses is similar to the ratio seen in humans (FIG. 16).

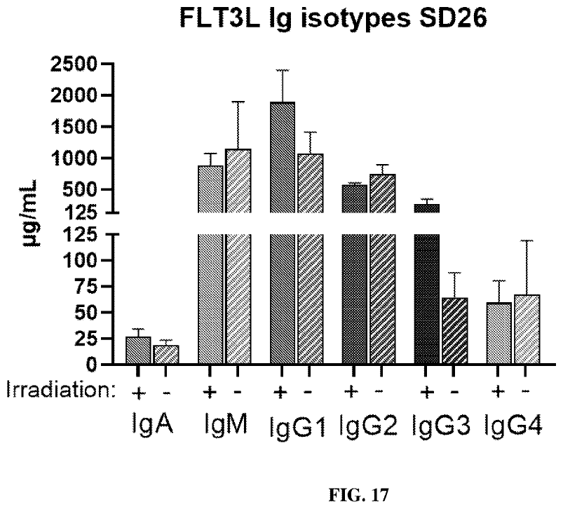

NSG-Flt3^{null} (hu-FLT3L) mice were then engrafted with 4 million PBMC from donor 0595 with or without 100 cGy of irradiation and bled once a week. Serum was analyzed for human IgM, IgA and the four IgG subclasses: IgG1, IgG2, IgG3, IgG4. The four IgG subclasses were quantified at a similar ratio as human levels and approaching the range seen in human adults (IgG1, 2.80-8.00 mg/ml; IgG2, 1.15-5.70 mg/mL; IgG3, 0.24-1.25 mg/mL; IgG4, 0.052-1.25 mg/mL) (FIG. 17). Normal human adult range for IgA is 0.8-3.0 mg/mL and for IgM is −0.4-2.5 mg/mL).

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1. A humanized immunodeficient mouse comprising human myeloid cells and human lymphoid cells, wherein the human lymphoid cells comprise human B cells that produce circulating immunoglobulin (Ig), and wherein the genome of the mouse comprises (a) a null mutation in an endogenous Flt3 allele and (b) a nucleic acid encoding a human FLT3L protein.

2.-19. (canceled)

20. A method of producing a humanized immunodeficient mouse, the method comprising:
administering human peripheral blood mononuclear cells (huPBMCs) to an immunodeficient mouse, wherein the immunodeficient mouse
(a) comprises a nucleic acid encoding a human FLT3L protein and expresses a detectable level of human FLT3L protein and
(b) comprise a null mutation in an endogenous Flt3 allele and does not express a detectable level of mouse FLT3 protein,
thereby producing a humanized immunodeficient mouse comprising human myeloid cells and human lymphoid cells that comprise human B cells that produce circulating immunoglobulin (Ig).

21. The method of claim 20, further comprising:
subjecting the immunodeficient mouse to a myeloablative treatment prior to administering the huPBMCs.

22. (canceled)

23. The method of claim 20, wherein the administering comprises administering at least 1 million of the huPBMCs to the immunodeficient mouse.

24. (canceled)

25. The method of claim 20 further comprising:
administering a target drug to the humanized immunodeficient mouse; and
assaying a biological sample from the mouse for a characteristic of an anti-drug antibody (ADA) response.

26.-27. (canceled)

28. The method of claim 25, wherein the characteristic is selected from ADA titer, neutralizing capacity, binding affinity, and isotype.

29. The method of claim 25, wherein the target drug is selected from vaccines, antibodies, recombinant protein therapeutics (e.g., growth factors), cell-based therapies (e.g., chimeric antigen receptor (CAR)-T cell, TCR-engineered T cell, tumor-infiltrating lymphocyte (TIL), and regulatory T cell (Treg) therapies), DNA-based (e.g., gene, antisense oligonucleotide) therapies, and RNA-based (e.g., RNAi and mRNA) therapies.

30. The method of claim 20 further comprising:
administering a human therapeutic agent to the humanized immunodeficient mouse; and
assaying a biological sample from the mouse for immunogenicity.

31.-32. (canceled)

33. The method of claim 30, wherein the human therapeutic agent is selected from vaccines, antibodies, recombinant protein therapeutics (e.g., growth factors), cell-based therapies (e.g., chimeric antigen receptor (CAR)-T cell, TCR-engineered T cell, tumor-infiltrating lymphocyte (TIL), and regulatory T cell (Treg) therapies), DNA-based (e.g., gene, antisense oligonucleotide) therapies, and RNA-based (e.g., RNAi and mRNA) therapies.

34. The method of claim 30, wherein the assaying comprises characterizing plasma or human B cell function.

35. The method of claim 30, wherein the assaying comprises detecting antigen-specific human T cells and/or activation markers on human B cells, human T cells, and/or human myeloid cells.

36. The method of claim 20 further comprising:
administering to the humanized immunodeficient mouse a chemical agent that induces a human autoimmune response characteristic of a human autoimmune disease;
administering a human therapeutic agent to the humanized immunodeficient mouse; and
assaying a biological sample from the mouse for an inflammatory response.

37.-38. (canceled)

39. The method of claim 36, wherein the chemical agent is selected from 2,4,6-Trinitrobenzene sulfonic acid (TNBS) (e.g., in ethanol), dextran sulfate sodium (DSS), oxazolone, type II collagen, myelin, and pristane.

40. The method of claim 36, wherein the autoimmune disease is selected from systemic lupus erythematosus, inflammatory bowel disease, multiple sclerosis, type 1 diabetes mellitus, psoriasis, and rheumatoid arthritis.

41. The method of claim 36, wherein the assaying comprises (a) recording body condition score, fecal score, and/or body weight over time; and/or (b) euthanizing the mouse and weighing the colon of the mouse and/or measuring the length of the colon of the mouse.

42. (canceled)

43. The method of claim 20 further comprising:
administering to the humanized immunodeficient mouse of an agent that facilitates sensitization in the mouse;
administering a human therapeutic agent to the humanized immunodeficient mouse;
challenging the humanized immunodeficient mouse; and
assaying a biological sample from the mouse for an inflammatory response.

44.-45. (canceled)

46. The method of claim 43, wherein the agent is selected from 2,4-Dinitro-1-fluorobenzene (DNFB), oxazolone, and keyhole limpet hemocyanin (KLH).

47. The method of claim 43, wherein the assaying comprises measuring human cytokine and/or chemokine levels.

48. The method of claim 20 further comprising:
administering a human therapeutic agent to the humanized immunodeficient mouse; and
assessing a biological sample from the mouse for human antibodies that bind specifically to the human therapeutic agent.

49.-52. (canceled)
